SEQUENCE LISTING (79 sequences)

SEQ ID NO: 1 - DNA, 189 bp, Artificial Sequence
  Codon-optimised pedA gene
atgaagaagattgagaagctgaccgaaaaggaaatggcgaacatcattggaggcaagtactatggcaatggtgtgacctgtggcaaacactcctgctctgtcgattggggcaaagccaccacgtgcatcatcaacaacggtgcaatggcatgggctactggaggtcatcaggggaaccacaaatgctaa

SEQ ID NO: 2 - protein, 62 aa, Pediococcus acidilactici
MKKIEKLTEKEMANIIGGKYYGNGVTCGKHSCSVDWGKATTCIINNGAMAWATGGHQGNHKC

SEQ ID NO: 3 - protein, 44 aa, Pediococcus acidilactici
KYYGNGVTCGKHSCSVDWGKATTCIINNGAMAWATGGHQGNHKC

SEQ ID NO: 4 - protein, 18 aa, Pediococcus acidilactici
MKKIEKLTEKEMANIIGG

SEQ ID NO: 5 - DNA, 525 bp, Artificial Sequence
  Codon-optimised pedC gene
atgagcaagaagttctggtccaacatctttctggctcttggtgtgttcctggcgtttgctggggttgcaaccatctcagtttccgctgatagctctgccaccattgagtccaacacctcctcgaagatcatcgatggagcgacgtacgaagagaacattcgtggtgtcattcccattaccctcactcagtatctccacaaagcacaaacgggcgaaaagttcatcgtctttgttggcttcaaggaatgcgtgcattgtcgcaaattcagccctgtgatgaagcagtaccttcagcagtctcaacacccgatctactacctggattatggcaacaatggctccttctctatggcttcacagaagcaaatcaccgacttctactccacctttgcaaccccaatgtccttcatgggaactccaacagtagccctgttggacaatggcaaagtcgtgtcgatgactgccggtgatgacaccacactgtccgatttgcagcagatcaccgcagactacaacaaccagtag

SEQ ID NO: 6 - protein, 174 aa, Pediococcus acidilactici
MSKKFWSNIFLALGVFLAFAGVATISVSADSSATIESNTSSKIIDGATYEENIRGVIPITLTQYLHKAQTGEKFIVFVGFKECVHCRKFSPVMKQYLQQSQHPIYYLDYGNNGSFSMASQKQITDFYSTFATPMSFMGTPTVALLDNGKVVSMTAGDDTTLSDLQQITADYNNQ

SEQ ID NO: 7 - DNA, 2175 bp, Artificial Sequence
  Codon-optimised pedD gene
atgtggacacagaagtggcacaagtactacacagcgcaagtagacgagaatgattgcggcctcgcagccctgaacatgatcttgaagtactacggttccgattacatgcttgcccatcttcgccagctggctaaaaccacagctgacggtactaccgtgctgggactggtgaaagcagccaagcacttgaacttgaatgctgaagctgttcgtgcggacatggacgcgctgaccgcgtcgcaactgcccctgcctgtgatcgttcacgttttcaagaagaacaagctgccacactactacgtggtctaccaggtgactgagaatgatctcatcattggcgatccggatccgaccgtgaaaactaccaagatctccaagtcccagtttgctaaggagtggacccagattgctattatcatcgctccgaccgtcaaatacaagcccatcaaggaaagccgtcacacgcttattgaccttgtccctctccttatcaaacagaaacgcctgattggactgatcattaccgcagccgcaatcaccaccctgatctctattgcaggcgcctacttctttcagctgatcattgacacatatctcccacacctgatgaccaaccgcctctctctcgtcgcaattgggcttattgtcgcatacgcattccaggccatcattaactatatccagtccttctttacgatcgtcctgggtcagcggctcatgatcgacattgtgctcaagtatgtccaccacctcttcgacctccctatgaacttctttaccactcggcatgtaggtgaaatgacgtcacgcttttccgatgcttccaaaatcattgacgctttggggtcaacgactctgaccctgttcctcgatatgtggatcctgttggctgttggactctttctggcgtatcagaacatcaacctgttcctgtgctcgctggtagttgttcccatctatatctccatcgtctggctcttcaagaaaaccttcaaccggcttaaccaggacactatggaatccaatgctgtccttaacagcgcgatcattgaatctctgagcggtatcgaaaccatcaagtcgcttactggagaggcgaccacgaagaagaagatcgataccctgttcagcgatcttctccacaagaatctggcctatcaaaaggccgatcagggtcaacaggccatcaaagccgcgacaaagcttatcctgacgatcgtgatcctctggtggggcaccttctttgtcatgcgtcatcagctgtccttgggccagctgttgacctacaatgcacttcttgcgtacttcttgacgccactcgagaacatcattaacctccaaccaaaactgcaagctgctcgcgttgctaacaatcgcttgaacgaggtttacctggtagagtcagagttctccaaaagccgagaaatcactgcacttgaacagttgaacggtgatattgaggtcaaccacgtaagcttcaattacggctactgctcgaacattctggaagatgtgtctttgacgatcccgcaccatcagaaaatcaccattgttggcatgtctggttccggcaaaaccaccctggcgaagttgcttgtgggcttcttcgaaccacaagaacagcatggcgagattcaaatcaaccaccacaacatctccgacatctcccgaaccattctccgccagtatatcaactacgtcccacaagaaccgttcatcttttccggttccgttctcgaaaacctgttgctgggctcccgccctggcgtgactcaacagatgatcgaccaggcttgttcattcgccgagattaagaccgatatcgagaatctccctcagggataccatactcgcctttccgagtctggctttaacctgagcggtggacagaagcagcgtctttcgatcgcacgtgcactgttgtccccagcacagtgtttcatctttgacgaatctacctcgaatctggacaccatcaccgagcacaaaatcgtgtccaagctcttgttcatgaaggacaaaaccatcattttcgtcgcacatcgcttgaatatcgcctctcagactgataaggtggttgttctggatcacggcaagattgtggagcaagggtcacaccgtcaactgctgaactacaacgggtattacgcacgcctcattcataaccaggaataa

SEQ ID NO: 8 - protein, 724 aa, Pediococcus acidilactici
MWTQKWHKYYTAQVDENDCGLAALNMILKYYGSDYMLAHLRQLAKTTADGTTVLGLVKAAKHLNLNAEAVRADMDALTASQLPLPVIVHVFKKNKLPHYYVVYQVTENDLIIGDPDPTVKTTKISKSQFAKEWTQIAIIIAPTVKYKPIKESRHTLIDLVPLLIKQKRLIGLIITAAAITTLISIAGAYFFQLIIDTYLPHLMTNRLSLVAIGLIVAYAFQAIINYIQSFFTIVLGQRLMIDIVLKYVHHLFDLPMNFFTTRHVGEMTSRFSDASKIIDALGSTTLTLFLDMWILLAVGLFLAYQNINLFLCSLVVVPIYISIVWLFKKTFNRLNQDTMESNAVLNSAIIESLSGIETIKSLTGEATTKKKIDTLFSDLLHKNLAYQKADQGQQAIKAATKLILTIVILWWGTFFVMRHQLSLGQLLTYNALLAYFLTPLENIINLQPKLQAARVANNRLNEVYLVESEFSKSREITALEQLNGDIEVNHVSFNYGYCSNILEDVSLTIPHHQKITIVGMSGSGKTTLAKLLVGFFEPQEQHGEIQINHHNISDISRTILRQYINYVPQEPFIFSGSVLENLLLGSRPGVTQQMIDQACSFAEIKTDIENLPQGYHTRLSESGFNLSGGQKQRLSIARALLSPAQCFIFDESTSNLDTITEHKIVSKLLFMKDKTIIFVAHRLNIASQTDKVVVLDHGKIVEQGSHRQLLNYNGYYARLIHNQE

SEQ ID NO: 9 - protein, 54 aa, Lactococcus lactis
MKELSEKELRECVGGGTWDDIGQGIGRVAYWVGKAMGNMSDVNQASRINRKKKH

SEQ ID NO: 10 - protein, 39 aa, Lactococcus lactis
GTWDDIGQGIGRVAYWVGKAMGNMSDVNQASRINRKKKH

SEQ ID NO: 11 - protein, 60 aa, Lactococcus lactis
MKNNNNFFKGMEIIEDQELVSITGGKKWGWLAWVDPAYEFIKGFGKGAIKEGNKDKWKNI

SEQ ID NO: 12 - protein, 35 aa, Lactococcus lactis
KKWGWLAWVDPAYEFIKGFGKGAIKEGNKDKWKNI

SEQ ID NO: 13 - protein, 56 aa, Lactobacillus plantarum
MLQFEKLQYSRLPQKKLAKISGGFNRGGYNFGKSVRHVVDAIGSVAGIRGILKSIR

SEQ ID NO: 14 - protein, 33 aa, Lactobacillus plantarum
FNRGGYNFGKSVRHVVDAIGSVAGIRGILKSIR

SEQ ID NO: 15 - protein, 52 aa, Lactobacillus plantarum
MKKFLVLRDRELNAISGGVFHAYSARGVRNNYKSAVGPADWVISAVRGFIHG

SEQ ID NO: 16 - protein, 34 aa, Lactobacillus plantarum
VFHAYSARGVRNNYKSAVGPADWVISAVRGFIHG

SEQ ID NO: 17 - protein, 55 aa, Lactobacillus plantarum
MTVNKMIKDLDVVDAFAPISNNKLNGVVGGGAWKNFWSSLRKGFYDGEAGRAIRR

SEQ ID NO: 18 - protein, 25 aa, Lactobacillus plantarum
GAWKNFWSSLRKGFYDGEAGRAIRR

SEQ ID NO: 19 - protein, 57 aa, Lactobacillus plantarum
MKIKLTVLNEFEELTADAEKNISGGRRSRKNGIGYAIGYAFGAVERAVLGGSRDYNK

SEQ ID NO: 20 - protein, 32 aa, Lactobacillus plantarum
RRSRKNGIGYAIGYAFGAVERAVLGGSRDYNK

SEQ ID NO: 21 - protein, 47 aa, Lactobacillus plantarum
MDKFEKISTSNLEKISGGDLTTKLWSSWGYYLGKKARWNLKHPYVQF

SEQ ID NO: 22 - protein, 29 aa, Lactobacillus plantarum
DLTTKLWSSWGYYLGKKARWNLKHPYVQF

SEQ ID NO: 23 - protein, 55 aa, Lactobacillus plantarum
MNNLNKFSTLGKSSLSQIEGGSVPTSVYTLGIKILWSAYKHRKTIEKSFNKGFYH

SEQ ID NO: 24 - protein, 34 aa, Lactobacillus plantarum
SVPTSVYTLGIKILWSAYKHRKTIEKSFNKGFYH

SEQ ID NO: 25 - protein, 75 aa, Lactococcus lactis
MKNQLNFNIVSDEELSEANGGKLTFIQSTAAGDLYYNTNTHKYVYQQTQNAFGAAANTIVNGWMGGAAGGFGLHH

SEQ ID NO: 26 - protein, 54 aa, Lactococcus lactis
KLTFIQSTAAGDLYYNTNTHKYVYQQTQNAFGAAANTIVNGWMGGAAGGFGLHH

SEQ ID NO: 27 - protein, 68 aa, Lactococcus lactis
MKNQLNFNIVSDEELAEVNGGSLQYVMSAGPYTWYKDTRTGKTICKQTIDTASYTFGVMAEGWGKTFH

SEQ ID NO: 28 - protein, 47 aa, Lactococcus lactis
SLQYVMSAGPYTWYKDTRTGKTICKQTIDTASYTFGVMAEGWGKTFH

SEQ ID NO: 29 - protein, 70 aa, Lactococcus garvieae
MENKNYTVLSDEELQKIDGGEYHLMNGANGYLTRVNGKYVYRVTKDPVSAVFGVISNGWGSAGAGFGPQH

SEQ ID NO: 30 - protein, 50 aa, Lactococcus garvieae
EYHLMNGANGYLTRVNGKYVYRVTKDPVSAVFGVISNGWGSAGAGFGPQH

SEQ ID NO: 31 - protein, 57 aa, Lactococcus lactis
MSTKDFNLDLVSVSKKDSGASPRITSISLCTPGCKTGALMGCNMKTATCNCSIHVSK

SEQ ID NO: 32 - protein, 34 aa, Lactococcus lactis
ITSISLCTPGCKTGALMGCNMKTATCNCSIHVSK

SEQ ID NO: 33 - protein, 51 aa, Lactococcus lactis
MKEQNSFNLLQEVTESELDLILGAKGGSGVIHTISHECNMNSWQFVFTCCS

SEQ ID NO: 34 - protein, 27 aa, Lactococcus lactis
KGGSGVIHTISHECNMNSWQFVFTCCS

SEQ ID NO: 35 - protein, 56 aa, Bacillus subtilis
MSKFDDFDLDVVKVSKQDSKITPQWKSESLCTPGCVTGALQTCFLQTLTCNCKISK

SEQ ID NO: 36 - protein, 32 aa, Bacillus subtilis
WKSESLCTPGCVTGALQTCFLQTLTCNCKISK

SEQ ID NO: 37 - protein, 56 aa, Staphylococcus epidermidis
MENKKDLFDLEIKKDNMENNNELEAQSLGPAIKATRQVCPKATRFVTVSCKKSDCQ

SEQ ID NO: 38 - protein, 30 aa, Staphylococcus epidermidis
SLGPAIKATRQVCPKATRFVTVSCKKSDCQ

SEQ ID NO: 39 - protein, 52 aa, Staphylococcus epidermidis
MEAVKEKNDLFNLDVKVNAKESNDSGAEPRIASKFICTPGCAKTGSFNSYCC

SEQ ID NO: 40 - protein, 22 aa, Staphylococcus epidermidis
IASKFICTPGCAKTGSFNSYCC

SEQ ID NO: 41 - protein, 55 aa, Staphylococcus epidermidis
MKKELFDLNLNKDIEAQKSDLNPQSASIVKTTIKASKKLCRGFTLTCGCHFTGKK

SEQ ID NO: 42 - protein, 31 aa, Staphylococcus epidermidis
SASIVKTTIKASKKLCRGFTLTCGCHFTGKK

SEQ ID NO: 43 - protein, 56 aa, Bacillus phage SPBeta
MEKLFKEVKLEELENQKGSGLGKAQCAALWLQCASGGTIGCGGGAVACQNYRQFCR

SEQ ID NO: 44 - protein, 37 aa, Bacillus phage SPBeta
GLGKAQCAALWLQCASGGTIGCGGGAVACQNYRQFCR

SEQ ID NO: 45 - protein, 66 aa, Carnobacterium piscicola
MNSVKELNVKEMKQLHGGVNYGNGVSCSKTKCSVNWGQAFQERYTAGINSFVSGVASGAGSIGRRP

SEQ ID NO: 46 - protein, 31 aa, Carnobacterium piscicola
WGQAFQERYTAGINSFVSGVASGAGSIGRRP

SEQ ID NO: 47 - protein, 47 aa, Kocuria varians
MTNAFQALDEVTDAELDAILGGGSGVIPTISHECHMNSFQFVFTCCS

SEQ ID NO: 48 - protein, 25 aa, Kocuria varians
GSGVIPTISHECHMNSFQFVFTCCS

SEQ ID NO: 49 - protein, 64 aa, Streptomyces sp. OH-4156
MRSEMTLTSTNSAEALAAQDFANTVLSAAAPGFHADCETPAMATPATPTVAQFVIQGSTICLVC

SEQ ID NO: 50 - protein, 22 aa, Streptomyces sp. OH-4156
ATPATPTVAQFVIQGSTICLVC

SEQ ID NO: 51 - protein, 52 aa, Staphylococcus gallinarum
MEAVKEKNELFDLDVKVNAKESNDSGAEPRIASKFLCTPGCAKTGSFNSYCC

SEQ ID NO: 52 - protein, 22 aa, Staphylococcus gallinarum
IASKFLCTPGCAKTGSFNSYCC

SEQ ID NO: 53 - protein, 68 aa, Bacillus sp. strain HIL Y-85,54728
MSQEAIIRSWKDPFSRENSTQNPAGNPFSELKEAQMDKLVGAGDMEAACTFTLPGGGGVCTLTSECIC

SEQ ID NO: 54 - protein, 20 aa, Bacillus sp. strain HIL Y-85,54728
CTFTLPGGGGVCTLTSECIC

SEQ ID NO: 55 - protein, 64 aa, Actinoplanes garbadinensis
MSALAIEKSWKDVDLRDGATSHPAGLGFGELTFEDLREDRTIYAASSGWVCTLTIECGTVICAC

SEQ ID NO: 56 - protein, 19 aa, Actinoplanes garbadinensis
SSGWVCTLTIECGTVICAC

SEQ ID NO: 57 - protein, 78 aa, Streptoverticillium griseoverticillatum
MTASILQQSVVDADFRAALLENPAAFGASAAALPTPVEAQDQASLDFWTKDIAATEAFACRQSCSFGPFTFVCDGNTK

SEQ ID NO: 58 - protein, 19 aa, Streptoverticillium griseoverticillatum
CRQSCSFGPFTFVCDGNTK

SEQ ID NO: 59 - protein, 77 aa, Streptoverticillium griseoverticillatum
MTASILQSVVDADFRAALIENPAAFGASTAVLPTPVEQQDQASLDFWTKDIAATEAFACKQSCSFGPFTFVCDGNTK

SEQ ID NO: 60 - protein, 19 aa, Streptoverticillium griseoverticillatum
CKQSCSFGPFTFVCDGNTK

SEQ ID NO: 61 - protein, 19 aa, Streptomyces sp. strain A647P-2
CVQSCSFGPLTWSCDGNTK

SEQ ID NO: 62 - protein, 44 aa, Enterococcus faecalis
GDVQAETTPVCAVAATAAASSAACGWVGGGIFTGVTVVVSLKHC

SEQ ID NO: 63 - protein, 68 aa, Enterococcus faecalis
MENLSVVPSFEELSVEEMEAIQGSGDVQAETTPVCAVAATAAASSAACGWVGGGIFTGVTVVVSLKHC

SEQ ID NO: 64 - protein, 27 aa, Enterococcus faecalis
GDVQAETTPACFTIGLGVGALFSAKFC

SEQ ID NO: 65 - protein, 63 aa, Enterococcus faecalis
MLNKENQENYYSNKLELVGPSFEELSLEEMEAIQGSGDVQAETTPACFTIGLGVGALFSAKFC

SEQ ID NO: 66 - protein, 67 aa, Staphylococcus aureus
MKNELGKFLEENELELGKFSESDMLEITDDEVYAAGTPLALLGGAATGVIGYISNQTCPTTACTRAC

SEQ ID NO: 67 - protein, 62 aa, Staphylococcus aureus
MKSSFLEKDIEEQVTWFEEVSEQEFDDDIFGACSTNTFSLSDYWGNKGNWCTATHECMSWCK

SEQ ID NO: 68 - protein, 63 aa, Streptococcus mutans
MSNTQLLEVLGTETFDVQEDLFAFDTTDTTIVASNDDPDTRFKSWSLCTPGCARTGSFNSYCC

SEQ ID NO: 69 - protein, 22 aa, Streptococcus mutans
FKSWSLCTPGCARTGSFNSYCC

SEQ ID NO: 70 - protein, 8 aa, Artificial Sequence
  Class IIA bacteriocin consensus sequence
  X (7)..(7): Any amino acid
YGNGGVXC

SEQ ID NO: 71 - DNA, 174 bp, Artificial Sequence
  Codon-optimised nisZ gene
atgtccaccaaagacttcaacctcgatcttgtgtctgtctccaagaaggattctggagcttcacctcgcattacctccatctccctgtgtacaccaggctgtaagactggtgccttgatgggctgcaacatgaaaaccgcaacgtgcaattgcagcatccacgtttcgaagtaa

SEQ ID NO: 72 - DNA, 2982 bp, Artificial Sequence
  Codon-optimised nisB gene
atgatcaaaagctctttcaaggctcagccgtttctggtgcgtaacaccattctgtctccgaacgacaaacgttctttcaccgaatacacccaggtgatcgaaaccgtgagcaaaaacaaagtctttctggaacagctgctgctggctaacccgaaactgtataatgttatgcagaaatacaacgcgggtctgctgaaaaagaagcgcgtaaagaaactgttcgagagcatctataaatactataagcgcagctacctgcgtagcaccccattcggtctgttctccgaaacctctattggcgtattttctaagtcttcccagtataagctgatgggtaagactaccaagggcatccgtctggacactcagtggctgattcgcctggtacacaaaatggaagttgacttctccaaaaaactgtccttcacccgtaacaacgccaattataaatttggcgaccgtgtgttccaggtttatactatcaactcctccgagctggaagaagtgaacattaaatataccaacgtatatcagatcatttccgaattttgcgaaaatgactaccagaaatacgaagacatttgcgaaaccgttaccctgtgctacggcgacgaataccgtgaactgtctggccagtatctgggcagcctgattgtaaaccactacctgatcagcaatctgcaaaaagacctgctgagcgatttctcctgggataccttcctgaccaaagtggaagcgattgacgaggataaaaaatatatcatcccgctgaagaaggtacagaaattcattcaggagtactccgaaatcgaaatcggtgagggtattgaaaaactgaaggaaatttaccaggagatgagccagattctggaaaacgacaactacattcaaatcgatctgatttccgactccgagatcaactttgatgttaaacagaaacaacagctggaacacctggcggagtttctgggtaacactaccaagtccgtgcgccgtacttatctggatgattacaaggataaatttatcgagaaatacggcgtagaccaggaagttcaaatcactgaactgtttgacagcacctttggcatcggtgcgccgtataactacaaccacccgcgtaacgacttctacgagtccgagccgtctactctgtattactctgaagaagaacgtgaaaaatatctgtctatgtatgtggaagcaattaaaaaccacaacgttattaatctggatgacctggaatctcactaccaaaaaatggacctggaaaaaaaatctgaactgcaaggcctggagctgtttctgaacctggcaaaagaatatgaaaaagatattttcatcctgggtgacatcgtaggtaacaataatctgggtggtgcatctggtcgtttctctgcactgtccccggagctgaccagctaccaccgtaccatcgttgactctgtggaacgcgagaacgaaaacaaagaaatcaccagctgcgaaatcgtattcctgcctgaaaacatccgtcatgccaacgttatgcacaccagcatcatgcgtcgtaaagtgctgccattcttcacgtccacttcccacaacgaagttcagctgactaacatttacattggcattgacgaaaaagaaaaattctatgcgcgtgacattagcacccaggaagtgctgaagttctacatcactagcatgtacaacaaaaccctgttttctaacgaactgcgttttctgtacgaaatcagcctggacgataaattcggtaacctgccgtgggaactgatctatcgtgatttcgactatatcccgcgcctggtttttgacgaaatcgtaattagcccggccaaatggaaaatttggggtcgtaacgtaaacaacaagatgactatccgtgagctgattcaaagcaaagagattccgaaagagttctatattgttaacggtgacaacaaagtgtatctgtcccaggaaaacccgctggatatggaaattctggaaagcgccatcaaaaaatctagcaaacgtaaagacttcattgaactgcaagaatacttcgaagacgaaaacatcatcaacaagggtcagaaaggtcgtgtggcggacgtagtggtgccattcatccgtactcgtgcactgggtaacgaaggtcgtgcttttatccgtgaaaagcgcgtgtctgttgaacgccgtgagaaactgccgtttaacgagtggctgtatctgaagctgtacatctccatcaaccgtcagaatgagttcctgctgagctatctgccggacattcagaaaatcgtggcaaacctgggtggtaacctgttttttctgcgttacaccgacccgaagcctcacattcgtctgcgcatcaaatgcagcgacctgtttctggcgtacggctctattctggaaatcctgaaacgtagccagaaaaaccgtattatgtccaccttcgatatttccatttatgatcaggaagtcgagcgttatggtggtttcgataccctggaactgtctgaagcaatcttctgcgcggattctaaaatcatcccgaacctgctgaccctgattaaagataccaacaacgactggaaagtcgatgacgtttccatcctggttaactacctgtacctgaagtgctttttccagaacgataacaagaagattctgaacttcctgaacctggtgtctccgaaaaaagtaaaggaaaacgtgaacgaaaaaatcgaacactacctgaaactgctgaaagtggacaacctgggcgatcagattttctacgacaaaaacttcaaggaactgaagcacgcaatcaagaacctgttcctgaaaatgatcgcgcaggactttgaactgcaaaaagtatattctatcatcgactccatcatccacgttcataacaaccgtctgatcggtatcgagcgtgataaagaaaaactgatctattacaccctgcaacgtctgttcgttagcgaggaatatatgaagtga

SEQ ID NO: 73 - DNA, 1803 bp, Artificial Sequence
  Codon-optimised nisT gene
atggatgaagtgaaggagttcacatcaaagcagttctttaacacgttgctcacttttccgtcaaccttgaagctcatcttccagctggagaaacgttatgcgatttaccttattgtcctgaacgccattaccgcatttgtacccctggcttctctgtttatctaccaagaccttattaactcagttctgggctctggtcggcatcttatctatatcatcatcatttacttcatcgtccaggtgatcactacggttctgggccaacttgaatcctatgttagcggtaagttcgacatgcgtctttcctattccatcaatatgcgcttgatgcgcacaacctcctccctggaactttcggattatgagcaggcagatatgtataacatcattgagaaggtcactcaggatagcacctataagccgttccagctcttcaatgcgattatcgtggttctgtcctcgttcatctcactgctctcctccttgttcttcattggcacttggaacatcggagttgccattctgctgctgattgtgcctgtgctgtccctcgtgctgtttctgcgcgtagggcagctggagttccttatccagtggcaacgcgcttctagcgaacgtgaaacctggtacattgtgtacttgcttacccatgacttcagcttcaaggagatcaagctcaataacatctcgaactactttatccacaaatttggcaagctgaagaaaggctttatcaatcaggatcttgccatcgcccgcaagaaaacctacttcaacatctttctggacttcatcctgaatttgatcaacatcttgaccatcttcgctatgatcctgtcagtccgagcaggcaaattgttgattgggaatctggtttccctcattcaggcgatttccaagattaacacctactctcagacgatgattcagaacatctacatcatctacaacacctccctcttcatggaacagttgttcgaattcctcaaacgcgaatcagtggttcacaagaaaatcgaggatacagagatctgcaatcaggatatcggaacggtaaaagtcatcaacctgtcctacgtctaccctaactccaatgcctttgcactcaagaacatcaacctctctttcgagaagggtgaattggcggctattgtgggtaagaacggttctggcaaatcgaccctcgttaagatcattagcggcctctatcaacccactatgggcattattcagtacgacaagatgcgttcctcgctcatgccagaggagttttaccagaagaacatctcggtgttgttccaggacttcgtgaaatacgagcttaccatccgcgaaaacattggtctgagcgacctgtcctcccagtgggaagatgagaaaatcatcaaagtccttgacaacctcggtcttgacttcctcaagaccaacaaccagtacgttctggacacccagctggggaattggttccaagaaggacgccaattgagcggaggtcaatggcagaagatcgcactggcacggactttcttcaagaaggcgtctatctacattctggacgaaccatccgctgcactcgatccagtagcagagaaggagatcttcgactactttgtcgccctttccgaaaacaatatctccatcttcatttctcacagccttaatgctgctcgcaaagctaacaagatcgtcgttatgaaggatggccaagtcgaagatgtgggatctcacgatgtcctgctgcgacgttgtcagtactaccaagaactgtactattccgagcaatacgaagataacgacgagtaa

SEQ ID NO: 74 - DNA, 1245 bp, Artificial Sequence
  Codon-optimised nisC gene
atgaataagaagaacatcaaacggaacgttgaaaagatcattgctcagtgggatgaacgcacacgcaagaacaaagaaaacttcgactttggcgaactgacgctttccaccggtttgccgggaattatcctcatgcttgccgagttgaagaacaaggataacagcaagatctaccagaagaagattgacaactatatcgagtacattgtcagcaaactgtccacctatggtttgttgaccggttcgctctactctggagctgccggaatcgctctgtctatcctccacctccgtgaggatgacgagaagtacaagaatcttctggattctctgaaccgctacatcgagtacttcgttatcgaaaagatcgagggcttcaatctggagaacatcactccacccgactacgatgtgatcgaaggtctgtcagggattctgtcatacttgcttctcatcaacgatgaacagtacgacgatctcaagatcctgatcatcaactttctgtcgaatctcaccaaggagaacaagggtttgatctcgctctacatcaaaagcgagaatcagatgagccaatcagaatcggaaatgtatcctctgggttgtctgaacatgggccttgctcatggcctggctggagcagggtgcattcttgcatacgcgcacattaaggggtattccaacgaagcatccctgtccgcacttcagaaaatcatcttcatctacgaaaagttcgaactcgaacgcaagaatcagtttctgtggaaagacggtctggtagccgatgaacttaagaaagagaaagtgattcgagaagcgtccttcattcgtgatgcatggtgctatggtggcccaggcatttccttgctttacctctatggaggcctcgcgcttgataacgactacttcgtggataaagccgagaagatcctggagtctgcgatgcaacgcaagctggggatcgactcctacatcatctgtcacggctactcaggcctcattgagatttgctccttgttcaaacggttgctgaacacgaagaagttcgactcctacattgaggagttcaacgtcaattccgagcaaatcctggaagagtatggcgatgagtctggtactggctttctggaaggcatctccggatgcattcttgtcttgtccaagtttgaatactctatcaacttcacctattggcgtcaggcactgctcctgttcgacgactttctgaaaggcggtaaacgcaaataa

SEQ ID NO: 75 - DNA, 213 bp, Artificial Sequence
  Codon-optimised garQ gene
atggagaacaacaactacaccgtgttgtccgatgaagagcttcagaagatcgatggaggcgaataccacctgatgaatggtgccaatggctatctcactcgtgtcaacggcaagtacgtctatcgcgttaccaaagacccagtttccgcggtattcggcgtgattagcaacggttggggatctgctggtgcagggtttggccctcaacattaa

SEQ ID NO: 76 - DNA, 314 bp, Artificial Sequence
  Codon-optimised garI gene
tgtttacctccaagaagatcaaacgcgatgaacaggtgttcctgaaccttctgtacgacttcgtgctttctgagaacatcactgatcgtgaacggaaaatcggcttgttggccaagaaggacgtagagaacggcaaatacctccttgcagtcctgaacaaggttagctcatccatgcaaaaggaagcgatgaagaatggtctgtccattgatgcatccagcttctacaagaaactgggtccaatcatcacctccattgctcctattgggctgaatcgcggatctatgctcgtcaacaactcgtatctcgactaa

SEQ ID NO: 77 - DNA, 2148 bp, Artificial Sequence
  Codon-optimised garC gene
atgcggttcaagaagaagtactatacctcccaagtggatgagcgcgactgtggttgcgcagcactgtccatggtgctcaaaacgtatggaaccgaacagtcactggcatctatccgtctgttggctggtactacaattgagggtacttccgctctgggaatcaagaaggctgctgaacacctgaagttctccgtgcaagcctttcgcgcagacccaacactcttcgataccaagaagttgccttacccgttcatcgtccatgtcgtcaaagagaagaaataccctcactactacgttgtcaagtcgtcgaacaagaaacacattatcatcgcagatccagaccctaccgtcaagatccgtaaaatgacctgggaccagttttactccgagtggacaggcgtaaccttgttcatcgcaccacagccatcctacaaacccgtgaaggagaagtccgaatctttgttctcgttcatcccaatccttgctaagcagaagctgctggtgttccagatcattattgcgtcccttctggttacactgattaacatcattggcagctactacctccaaaacatcatcgacgagtatgtccccaatgccctgatgaaaactctgagcatcgtgtcgctcggtctgatggctacgtacttgattcagcagttcctcaactttgcgcagacctttctcctgactatcctggggcaacgtttggctatcgacgttatcctttcatacatccggcacatctttcagctcccgatgtccttcttttcgacccgacgtacgggtgaaatcactagccgcttttcagatgcgaactccatcatcgatgcgttggctagcgctattctgtccctctttctcgacgtaaccatcgttattctgactggcttggttctgggtttgcagaacatgtctctcttcttcctggtgcttttcagcattccgctctatgtccttatcatctttatcttcattccgttgttcgagaagcagaacaacgaagtcatgcaggcgaattccattctgaattcctcaatcatcgaggacatcaacggtatcgaaaccatcaaatccctgggctctgaagagattcgctatcagaaaattgatcgcgaattcgccgattacatgaagaaatcgttcacgcgccagaagtcggaggcattccagactgccctgaagtccggtctgcaactggttctgaacgtgttgattctttggtacggtgccactctggtgatgtcccagaaaatcaccctcggacaacttatcaccttcaatgctcttctgtcctacttcatgaccccactttctaacatcatcaatctccagaccaagctccaatctgccaaggtcgcgaaccatcggctgaatgaagtttacctggtggatagcgagtttgacacgaacaccgatgaggttatcctttccaactttgagatgcagctgatcggcattagctatcactacggctttgggcgtgacattctttctaacatgtcccttaacatcaaagaaaacgaaaagcttaccattgtcggaatgtctggctcaggcaaatccaccctcgttaagcttctcgtaaacttcttcgaacccacccaaggaaccattatcctgggcgggattgacatcaaacaaatggataaacaccagctgcgttctatcgtgtcatatttgcctcaacagccttacatcttcaccggcaccgtcttggagaaccttattctgggcgcgtctgaagatctctctcaggaagagattttccaggcagtagaggcagcagaaatccgatccgatatcgaacagctccaactgggctatcagacggagctgtcatccgatgccacaactctctcgggtggacagaagcaacgcattgcgctcgctcgcgcacttctgtctccagccaagatcctgatcttcgatgaggctaccagcaatctggacgttattaccgagaagaagattttgaagaatctcttcaaactggacaagaccatcatcttcatcgcccatcgcttgagcattgcagaaaagtccgaccgcatcattgtgatcgatcaggggaaaatcatggaagaaggctcccacttggaactgctttccaagaatggcttctacgcccagatctacaaccagtaa

SEQ ID NO: 78 - DNA, 1428 bp, Artificial Sequence
  Codon-optimised garD gene
gtgtttgataagcgactcttggagtcgtctgagctgtatgaaaagcgctacaagaatttctccgtcctcctcatctttccagtcgcactgttgtttatcggtctgttcgtgttcagcttcttcgctaagaaggagctgatcgtgacgaacattgcctccattgctccgaagaagatcatcagcaacattcaatccacctccaatacgcccattatcgaaaaccacctggttgaggggaaatcagtgcagtctaattcactgctcatcaagtacaacaacgactctgactctactcagattaccaccctgatggatcaaaagcaggaattcctcaacaagaaggagcaacttcagttgcttctgaattcgctgcactctgataccaaccagttcccaaacaccgattcctatggatacgaaaagacgttcgaaacctacaaggcacaagccgaatcccttcgcgaatcgatccagaaatcgaaccaagtggttgacgatcagaacaactccattgagaaccagaagtctgcaatcaccacgcagatttccaacgtaaacacccaaatcaatgcttacatcgacatccagaatgcagctagcaccaacagctcagtatcctccaacaatccgtacatcgctcagtataactcgtacttggaacagcgcaaggttctcgaagaatccatgaagaatcaggaaaagactgaagcctcctcaaaggccgacattacccagcagaaagaggccttgaaagcgcaattcatctccaacatctcctccaacattgacaaccttaagaatcagatccagacactggaggtccagaaatcctccctgagcagctccaactcttacgatcaatctcagaaaagccaactcctcgcgcttaaaactcaggcgctcactgctgcgaacaaagaactgtccgacatcaatagcacacttaccgagattggtggcaaaatctctctccagaaacaggccaaccaatacaacgcaatctttgctgagaaagagggtatcttgcatgttctgcctaacgtgctggagaacaaaaccttccaggtaggtactccactggcacagatctatcctgctctgaaggcgaagtcacaggtctatctgacttcatacattccctccacccagatttcaggcattaagcttggacagcgtgcacgcttcacggtgcagcaaaacttgcctaagcttgagatcctgactggaaccatcaagcagatcgattccgcaccaacagcccttaaggagggtaattcgtacaaagtgtcggcactggttaccttgaacgaacgtgatctctcctacatccggtatggcttggaaggcaagtttgtcgtcgttaccggccaaaagacctacttcaactactacctggacaaaatcaagggccaagattctctgaattaa

SEQ ID NO: 79 - protein, 31 aa, Bacillus subtilis
MKKLLTVMTMAVLTAGTLLLPAQSVTPAAHA

(19) United States
(12) Patent Application Publication
(10) Pub. No.: US 2022/0348615 A1
(43) Pub. Date: Nov. 3, 2022

(54) PRODUCTION OF BACTERIOCINS

(71) Applicants: Norwegian University of Science and Technology (NTNU), Trondheim (NO); UNIVERSITÄT ULM, Ulm (DE); NORWEGIAN UNIVERSITY OF LIFE SCIENCES, Ås (NO); DANMARKS TEKNISKE UNIVERSITET, Kongens Lyngby (DK); UNIVERSITÄT DES SAARLANDES, Saarbrucken (DE)

(72) Inventors: Gerd Michael SEIBOLD, Kongens Lyngby (DK); Christoph WITTMANN, Saarlouis (DE); Dzung DIEP, Oslo (NO); Nadav BAR, Trondheim (NO); Christian U. REIDEL, Ulm (DE); Dominik WEIXLER, Ulm (DE); Oliver GOLDBECK, Neu-Ulm (DE)

(73) Assignees: Norwegian University of Science and Technology (NTNU), Trondheim (NO); UNIVERSITÄT ULM, Ulm (DE); NORWEGIAN UNIVERSITY OF LIFE SCIENCES, Ås (NO); DANMARKS TEKNISKE UNIVERSITET, Kongens Lyngby (DK); UNIVERSITÄT DES SAARLANDES, Saarbrücken (DE)

(21) Appl. No.: 17/724,712

(22) Filed: Apr. 20, 2022

(30) Foreign Application Priority Data
Apr. 21, 2021 (GB) 2105707.0

(51) Int. Cl. C07K 14/34

(52) U.S. Cl. CPC C07K 14/34

(57) ABSTRACT

The present invention relates to the production of bacteriocins, and in particular the production of bacteriocins of class I or II by recombinant expression in coryneform bacteria as the bacterial cell host. Also provided are modified coryneform bacteria for use as production hosts for production of bacteriocins.

The project leading to this application has received funding from the Bio Based Industries Joint Undertaking (JU) under grant agreement No 790507. The JU receives support from the European Union's Horizon 2020 research and innovation programme and the Bio Based Industries Consortium.

### FIELD

The present disclosure and invention relate to the production of bacteriocins, and in particular the production of bacteriocins of class I or II by recombinant expression in a bacterial cell host. Provided herein is a method for bacteriocin production based on expression of bacteriocin production genes in coryneform bacteria, notably bacteria of the genus Corynebacterium or Brevibacterium. Also provided herein are modified bacteria of said genera for use as production hosts for production of bacteriocins, wherein said bacteria have been modified to express a class I or II bacteriocin polypeptide.

### BACKGROUND

Bacteriocins are proteinaceous or peptidic toxins produced by bacteria to inhibit the growth of other bacteria, usually, but not always, similar or closely related bacteria. In other words, bacteriocins are antimicrobial peptides produced by bacteria. They are structurally, functionally and ecologically diverse. Bacteriocins have been categorised or classified in various ways, including based on producing strain, killing mechanism or resistance mechanism, properties of the bacteriocin polypeptide such as size or chemical modifications, and genetics of the producing bacteria (whether encoded by large or small plasmids, or chromosomally etc.).

Bacteriocins of Gram-positive bacteria are typically classified into Class I, Class IIA/B/C/D, Class III and Class IV. The Class I bacteriocins are typically small peptide inhibitors which comprise extensive post-translational modifications and include nisin and other lantibiotics. Lantibiotics are characterised by the presence of the modified amino acid lanthionine in their structures. Class II bacteriocins are small, typically less than 10 kDa, heat-stable proteins sub-divided into at least 4 sub-classes, A to D.

Bacteriocins of Gram-negative bacteria, meanwhile, are typically classified by size. Microcins are usually less than 20 kDa, colicin-like bacteriocins (CLBs) or colicins are usually from 20 to 90 kDa, and tailocins or high molecular weight bacteriocins include larger, multi-subunit bacteriocins. Each of these classes may be further divided into various sub-classes based on structure, functional mechanism or other characteristics.

Bacteriocins have found application in various areas based on their anti-bacterial effects, including clinically as antibiotics, or as anti-microbial agents to prevent food spoilage, or contamination etc., e.g. as a food preservative, or as plant protection agents when expressed in plants to provide resistance against plant disease. For example, the Class IIa bacteriocin Pediocin PA-1 has potent anti-microbial activity against a range of Gram-positive bacteria, including important human pathogens such as Listeria monocytogenes. The Class I bacteriocin nisin has been used as a food preservative for processed cheese, meats and beverages.

Since bacteriocins may provide a useful source of antibiotics to address the growing problem of antibiotic resistance, as well as for other uses, and in view of their favourable properties such as heat stability, there is interest in new and improved ways of producing them. To date, bacteriocins tend to be obtained from their native producing strains. However, whilst native producers provide a source, for commercial-scale production this is not without problems, since native producers, such as Pediococcus acidilactici, the native producer of pediocin PA-1, tend to have specific growth media and growth condition requirements which can make production difficult or expensive. Further, the use of native producers tends to require different processes to be developed for different bacteriocin products. There is thus a need for a bacteriocin production processes, and particularly for a process, or production platform, which may readily be used or adapted for the production of different bacteriocins.

### SUMMARY

We propose herein a novel production process for bacteriocins which is based on the new and surprising use of coryneform bacteria, or bacteria from the genera Corynebacterium and Brevibacterium, as production hosts for the production of bacteriocins by recombinant expression of bacteriocin-producing genes.

It has surprisingly been found that Corynebacterium species may be used as efficient hosts for the production of bacteriocins, particularly various bacteriocins in the Classes I and II, as exemplified by nisin and pediocin/garvicin Q respectively. Bacteria of the genus Brevibacterium are closely related to Corynebacterium spp. and indeed certain species of Corynebacterium were previously classified as Brevibacterium sp. It is accordingly proposed herein that Brevibacterium spp. may have analogous utility, and that accordingly coryneform bacteria in general may be used.

One way to establish bacteriocin production in a Corynebacterium host is when the producer organism is not susceptible to the bacteriocin in question. It has been possible to identify strains or species of coryneform bacteria, notably Corynebacterium, which are not susceptible to bacteriocins it is desired to produce. Alternatively, the production process may be adapted such that the producer host organism is not affected by the bacteriocin. This may be achieved by introducing genes which confer resistance to, or protect the host from, the bacteriocin, and/or by expressing the bacteriocin as an inactive precursor, or by expressing in one host organism an individual polypeptide component of a multi-polypeptide bacteriocin, or fewer components than are required to make up a functional (i.e. active) bacteriocin product. In such a case, individual components may be separately expressed and recovered from the respective host organisms, and then used to reconstitute, or assemble, a functional bacteriocin protein.

Accordingly, a first aspect provided herein is a method of producing a bacteriocin, said method comprising:

(i) providing a modified coryneform bacterial strain into which has been introduced a heterologous nucleic acid molecule encoding a bacteriocin polypeptide;

(ii) culturing said modified strain under conditions suitable for expression of said bacteriocin polypeptide; and

(iii) optionally, harvesting said bacteriocin polypeptide produced in step (ii),

wherein the bacteriocin polypeptide is an inactive precursor, and/or said strain is not susceptible to said bacteriocin, and/or said bacteriocin polypeptide is a component polypeptide of a multi-peptide bacteriocin and said modified strain does not produce all other component polypeptides required to make up a functional bacteriocin.

As is set out below in more detail, the coryneform bacterial strain may further comprise at least one heterologous nucleotide sequence encoding a polypeptide for biosynthesis, modification or export of the bacteriocin to be produced, referred to herein more generally as a polypeptide for processing and/or transport of the bacteriocin. Such a further nucleotide sequence may be contained in the same or in different nucleic acid molecule. Accordingly, step (i) may alternatively be defined as (i) providing a modified coryneform bacterial strain into which has been introduced one or more heterologous nucleic acid molecules encoding a bacteriocin polypeptide, and optionally encoding one or more processing and/or transport proteins for production of said bacteriocin polypeptide.

The bacteriocin which is to be produced may be a bacteriocin of a Gram-positive bacterium, or of a Gram-negative bacterium. In particular embodiments, the bacteriocin may be a bacteriocin of Gram-positive bacteria. More particularly, the bacteriocin may be a Class I or Class II bacteriocin of a Gram-positive bacterium.

Accordingly, provided herein is a method of producing a Class I or Class II bacteriocin, said method comprising:

(i) providing a modified coryneform bacterial strain into which has been introduced a heterologous nucleic acid molecule encoding a Class I or Class II bacteriocin polypeptide;

(ii) culturing said modified strain under conditions suitable for expression of said bacteriocin polypeptide; and

(iii) optionally, harvesting said Class I or Class II bacteriocin polypeptide produced in step (ii),

wherein the bacteriocin polypeptide is an inactive precursor, and/or said strain is not susceptible to said bacteriocin, and/or said bacteriocin polypeptide is a component polypeptide of a multi-peptide bacteriocin and said modified strain does not produce all other component polypeptides required to make up a functional bacteriocin.

The strain may be of the genus Corynebacterium or Brevibacterium.

The bacterial strain may be naturally resistant to the bacteriocin, or it may be engineered, or modified, to be resistant, for example by introducing a nucleotide sequence, in the same or different nucleic acid molecule, which encodes one or more proteins which confer resistance, or which render the bacterial strain not, or less, susceptible to the bacteriocin. Such a protein may be an immunity protein for example. In one option the nucleotide sequence which encodes the protein conferring resistance or rendering the bacterial strain not, or less, susceptible to the bacteriocin, e.g. an immunity protein, may be introduced such that it is constitutively expressed. In another alternative, the bacterial strain before modification may constitutively express a gene which provides the bacterial strain with immunity (or reduced or lower susceptibility) to the bacteriocin. However, in the alternative, the modified bacterial strain may not contain a gene which provides the bacterial strain with immunity (or reduced or lower susceptibility) to the bacteriocin, i.e. neither the bacterial strain before modification nor the introduced nucleotide sequence contains such a gene. Thus, there is further provided a method in which the modified bacterial strain a) does not contain a gene which provides the bacterial strain with immunity (or reduced or lower susceptibility) to the bacteriocin, or b) contains a constitutively expressed gene which provides the bacterial strain with immunity (or reduced or lower susceptibility) to the bacteriocin. In instances when the bacterial strain does not have immunity to the bacteriocin, alternatives methods of avoiding the activity of the expressed polypeptide may be used as discussed herein.

Therefore, in an embodiment, when said bacteriocin is a Class I bacteriocin, the bacteriocin polypeptide is an inactive precursor, and when said bacteriocin is a class II bacteriocin, said strain is not susceptible to said bacteriocin and/or said bacteriocin polypeptide is a single polypeptide of a multi-peptide bacteriocin. Class II bacteriocins may also be produced as inactive precursors.

The strain may not be susceptible by virtue of lacking a functional receptor or target for the bacteriocin. This may be due to lack of the presence of a receptor or target protein, e.g. due to lack of expression of the protein, or due to lack of a functional receptor or target protein, for example as result of modification to remove or inactivate the receptor or target protein, e.g. by genetic engineering. In other words, the strain may be modified to reduce or remove (e.g. knock-out or knock-down) expression of a receptor or target protein. For certain bacteriocins, e.g. pediocin or other Class II bacteriocins, coryneform bacteria, e.g. species or strains of Corynebacterium or Brevibacterium, may be used which naturally lack a protein capable of acting as a receptor, or a target or binding protein, or which naturally do not express a functional receptor or target protein, for the bacteriocin to be produced.

In an embodiment, where the bacteriocin is a Class II bacteriocin, the modified strain of coryneform bacteria does not express a protein capable of acting as a receptor or target for the bacteriocin to be expressed, or, alternatively put, the bacteriocin to be produced.

The nucleic acid molecule which is introduced to modify the bacteria comprises a nucleotide sequence which encodes the bacteriocin polypeptide. The nucleic acid molecule may thus be regarded as comprising a gene (or gene sequence) encoding the bacteriocin polypeptide. More particularly, this gene may be regarded as representing a structural gene for the bacteriocin polypeptide.

Depending on the nature of the bacteriocin, the nucleic acid molecule may, as noted above, comprise one or more further nucleic acid molecules, which comprise one or more nucleotide sequences (or in other words, genes or gene sequences) which encode other proteins involved in the processing and/or transport of the bacteriocin. This may include proteins, e.g. enzymes, which are involved in modification of the expressed bacteriocin polypeptide, for example enzymes which modify the bacteriocin polypeptide, e.g. by post-translational modification. This may also include proteins involved in the export, e.g. secretion, of the bacteriocin. The nucleic acid molecule(s) may further comprise a nucleotide sequence encoding a protein which confers immunity to the bacteriocin (i.e. an immunity protein).

The nucleotide sequences, i.e. genes, may be codon-optimised for expression in coryneform bacteria, more particularly for expression in Corynebacterium sp. and/or Brevibacterium sp.

A further aspect provided herein is a strain of coryneform bacteria (more particularly, a bacterial strain of the genus Corynebacterium or Brevibacterium) which has been modified to express a bacteriocin polypeptide, wherein the bacteriocin polypeptide is an inactive precursor, and/or said strain is not susceptible to said bacteriocin, and/or said bacteriocin polypeptide is a component polypeptide of a multi-peptide bacteriocin and said modified strain does not produce all other component polypeptides required to make up a functional bacteriocin.

In still another aspect also provided herein is an expression vector capable of expressing a bacteriocin polypeptide in coryneform bacteria, e.g., in a bacterial strain of the genus Corynebacterium or Brevibacterium, said expression vector comprising a nucleic acid molecule comprising a synthetic operon comprising:

(i) a promoter controlling the expression of the following genes;

(ii) a structural gene encoding the bacteriocin polypeptide;

(iii) optionally, one or more genes encoding processing and/or transport proteins for production of said bacteriocin polypeptide; and/or

(iv) optionally, one or more genes which provide the bacterial strain with immunity to the bacteriocin;

wherein the nucleotide sequences of said genes are codon-optimised for expression in coryneform bacteria.

As noted above, the bacteriocin may be any bacteriocin, including of Gram-positive or Gram-negative bacteria. In particular embodiments the bacteriocin is of Gram-positive bacteria, and more particularly a Class I or Class II bacteriocin.

In some embodiments of these aspects, when said bacteriocin is a Class I bacteriocin, the bacteriocin polypeptide is an inactive precursor, and wherein when said bacteriocin is a Class II bacteriocin, said strain is not susceptible to said bacteriocin and/or said bacteriocin polypeptide is a single polypeptide of a multi-peptide bacteriocin. Alternatively, or additionally, the Class II bacteriocin may be produced as an inactive precursor.

### DETAILED DESCRIPTION

The methods, bacterial strains and expression vectors herein are based on the concept of using bacteria of the genus Corynebacterium or Brevibacterium as host cells for the production of bacteriocins. It has been found that, unexpectedly, coryneform bacteria of the genus Corynebacterium or Brevibacterium are suitable, and indeed good, hosts for the production of bacteriocins.

Bacteriocins are bacterial products that are toxic to other bacteria. However, the inhibitory spectrum of some bacteriocins is narrow, and appropriate species or strains of coryneform bacteria may be selected which are not susceptible to the bacteriocin to be produced. For example, Corynebacterium spp. or Brevibacterium spp. may be selected which are not susceptible to pediocin, or other Class II bacteriocins. Further, by modifying the bacteria and/or the production process such that the bacteria express a bacteriocin polypeptide which is not active, and/or to render the bacteria not susceptible to the bacteriocin product which is produced, bacteria of these genera may be used to produce a range of different bacteriocins. Thus, the bacteriocin in question may have an underlying toxicity to the bacteria, but despite this, the expression process or the bacteria may be modified to circumvent, or to avoid this toxicity, and render the bacteria useful as production hosts for the bacteriocin.

This has advantages, as bacteria of these genera have advantageous properties as production hosts. In particular, coryneform bacteria tend to have a broad substrate spectrum, and are able to grow using a variety of materials as nutrient sources, including waste materials and by-products of various industrial and manufacturing processes. They are not fastidious, and do not have strict nutrient or growth media requirements, as may be the case with many natural bacteriocin producers. Thus, for example waste sugars such as spent sulphite liquor, residual sugars of the pulp industry may be used. The growth of coryneform bacteria on various substrates has been well-studied, and procedures for large-scale growth have been developed. Thus, the present method may readily be up-scaled for production on a commercial scale. Advantageously, the bacteriocins may be produced as extracellular products which are secreted by the bacterial cells, which facilitates recovery of the produced bacteriocins. Coryneform bacteria have the advantage that low amounts of other products are secreted into the growth supernatant, simplifying purification, and facilitating recovery further. Coryneform bacteria also tend to have low extracellular protease activity, leading to a more stable product. Accordingly, the present methods and strains allow improved and optimised bacteriocin production to be achieved, allowing for the production rate to be increased several-fold.

The present inventors have devised a production platform for bacteriocins using coryneform bacteria as hosts which may applied to the production of different bacteriocins using the same organism as production host. As will be described in more detail below, in some embodiments, the native, or endogenous, protein export and transport machinery of the bacterial host may be used to achieve secretion of the expressed bacteriocin into the extracellular growth medium (supernatant). However, in other embodiments one or more genes encoding transport and export proteins may be introduced into the modified strains. In particular embodiments, a system has been developed which uses genes encoding export and transport proteins derived from a particular bacteriocin gene cluster as the basis for a generic transport and export system. That is, the generic system may be used for transport and export of the bacteriocin to be expressed, including different bacteriocins (i.e. the transport and export genes may be heterologous to the gene encoding the bacteriocin polypeptide, that is derived from a different source). Surprisingly, it has been found that these heterologous transport and export genes remain effective in the host cell, despite the different environment and in particular the different cell-wall structures. In particular Coryneform bacteria have an additional membrane permeability barrier (the mycolic acid layer) and a different membrane composition, but the heterologous molecules were still found to be effective. In this way a standardised bacteriocin production system may be developed, which allows different bacteriocins to be produced using the same production machinery. For example, vectors may be developed which allow a gene encoding a desired bacteriocin polypeptide to be introduced, for expression in conjunction with genes contained in the vector for the transport and export of the bacteriocin polypeptide. This is described further below.

The strain may be any strain or species of coryneform bacteria. Coryneform bacteria include bacteria belonging to the genus Corynebacterium or Brevibacterium. As noted above, Brevibacterium is closely related to Corynebacterium, and indeed certain Corynebacterium species were previously classified as Brevibacterium species. More particularly, coryneform bacteria may be defined as bacteria in the genus Corynebacterium, including bacteria formerly classified as Brevibacterium, and bacteria belonging to the genus Brevibacterium, particularly those closely related to the genus Corynebacterium.

In an embodiment, the bacterial strain may be a species selected from Corynebacterium glutamicum (also known as Brevibacterium lactofermentum), Corynebacterium efficiens, Corynebacterium ammoniagenes, Corynebacterium melassecola, Corynebacterium acetoacidophilum, Corynebacterium lilium, Corynebacterium casei, Corynebacterium stationis, Brevibacterium flavum, and Brevibacterium divaricatum.

Other representative species, which may be used include: C. acetoglutamicum, C. alkanolicum, C. callunae, C. herculis, B. immariophilum, B. roseum, B. saccharolyticum, B. thiogenitalis, B. album, B. cerinum,

Details of representative strains of such species are listed in US 2009/0286290 and U.S. Pat. No. 5,498,532, for example.

Strains of Corynebacterium glutamicum have been developed for various industrial uses, and any such strain may be used. For example, mention may be made of the type strain ATCC13032 or the strain R. Another representative strain is strain CR099, which is an early strain developed for use in production of amino acids. Such strains have been modified to remove genes unnecessary for the growth of the bacteria to produce amino acids. Such work led to the development of strain C1, another representative strain that may be used. (Baumgart et al. ACS Synthetic Biology 7.1 (2018): 132-144). Various strains are available from different culture collections and any available strain or derivative thereof may be used. Thus, mutants of available strains may be used.

Further, any other non-pathogenic, non-protease producing species or strains of the genus Corynebacterium or Brevibacterium which may be used, include strains of Corynebacterium efficiens, such as the type strain DSM44549, Corynebacterium ammoniagenes, such as strain ATCC6871, Corynebacterium melassecola strain ATCC17965, Brevibacterium flavum strain ATCC14067, Brevibacterium lactofermentum strain ATCC13869, and Brevibacterium divaricatum strain ATCC14020 strain. Some representatives of the species Corynebacterium glutamicum are also known in the art under other names. These include, for example strain ATCC13870, which was named Corynebacterium acetoacidophilum, strain DSM20137, which was called Corynebacterium lilium, strain ATCC17965, which was called Corynebacterium molassecola, strain ATCC14067, designated Brevibacterium flavum, strain ATCC13869, which was designated Brevibacterium lactofermentum, other strain ATCC14020, designated Brevibacterium divaricatum. The term “Micrococcus glutamicus” for Corynebacterium glutamicum was also common.

The methods, strains and vectors herein are of general applicability and may be applied to any bacteriocin, including any bacteriocin known or reported in the art, as well as bacteriocins yet to be discovered. Class I and II bacteriocins are bacteriocins produced by Gram-positive bacteria. These are generally low molecular weight antimicrobial peptides with usually fewer than 60 amino acids. They may vary in terms of size, structure, physico-chemical properties and inhibitory spectrum. However, the precise form and size of the bacteriocin is not critical to the methods, strains and vectors herein.

Class I bacteriocins are post-translationally modified peptides, many of which comprise extensive modifications. Thus, the encoded bacteriocin polypeptide expressed from the structural gene which encodes the bacteriocin polypeptide, i.e. the translation product, is subjected to modification of the constituent amino acids. The modified peptides may be linear or globular, are generally small in size, e.g. they may be less than 5 kDa in size, or may have less than 40 amino acids, and may contain lanthionine, β-methyl lanthionine, and/or dehydrated amino acids. This class includes the lantibiotics, as typified by the linear peptide nisin or the globular peptide mersacidin, which are characterized by comprising the modified amino acid lanthionine, labyrinthopeptins, such as globular peptide labyrinthopeptin A2, and sactibiotics, such as the globular peptide subtilosin A. Of particular interest herein are the lantibiotics.

Exemplary lantibiotics include nisin, bisin, lacticin, subtilin, epicidin, epidermin, epilancin, salvaricin, sublancin, carnocin, variacin, cypemycin, gallidermin, mersacidin, actagardine, cinnamycin, duramycin, ancovenin, cytolysin, staphylococcin and mutacin,

In an embodiment the class I bacteriocin is nisin. Four naturally occurring variants of nisin from Lactococcus lactis have been identified, nisin A, nisin Q, nisin F and nisin Z, which differ in one or more amino acid residues at position 15, 21, 27, and/or 30. The related peptide nisin U has been identified from Streptococcus uberis.

Class I bacteriocins are typically expressed as precursor polypeptides, or pre-peptides, which are inactive, and are proteolytically cleaved to activate the peptide and release an active bacteriocin polypeptide, the mature form of the polypeptide. In particular, the class I bacteriocin is expressed with a leader sequence (also referred to herein as the signal peptide, SP), which directs secretion of the polypeptide and which is cleaved, typically by a dedicated protease, to release the mature and active bacteriocin peptide.

By way of representative example, the sequences of various Class I bacteriocins with (i.e. precursor polypeptides) or without (i.e. mature polypeptides) leader sequences are provided below.

The amino acid sequence of the mature form of nisin Z is set out in SEQ ID NO. 32, and the amino acid sequence of the precursor comprising the leader sequence is set out in SEQ ID NO. 31.

The amino acid sequence of the mature form of lacticin is set out in SEQ ID NO. 34, and the precursor comprising the leader sequence is set out in SEQ ID NO. 33.

The amino acid sequence of the mature form of subtilin is set out in SEQ ID NO. 36, and the amino acid sequence of the precursor comprising the leader sequence is set out in SEQ ID NO. 35.

The amino acid sequence of the mature form of epicidin is set out in SEQ ID NO. 38, and the amino acid sequence of the precursor comprising the leader sequence is set out in SEQ ID NO. 37.

The amino acid sequence of the mature form of epidermin is set out in SEQ ID NO. 40, and the amino acid sequence of the precursor comprising the leader sequence is set out in SEQ ID NO. 39.

The amino acid sequence of the mature form of epilancin is set out in SEQ ID NO. 42, and the amino acid sequence of the precursor comprising the leader sequence is set out in SEQ ID NO. 41.

The amino acid sequence of the mature form of sublancin is set out in SEQ ID NO. 44, and the amino acid sequence of the precursor comprising the leader sequence is set out in SEQ ID NO. 43.

The amino acid sequence of the mature form of carnocin is set out in SEQ ID NO. 46, and the amino acid sequence of the precursor comprising the leader sequence is set out in SEQ ID NO. 45.

The amino acid sequence of the mature form of variacin is set out in SEQ ID NO. 48, and the amino acid sequence of the precursor comprising the leader sequence is set out in SEQ ID NO. 47.

The amino acid sequence of the mature form of cypemycin is set out in SEQ ID NO. 50, and the amino acid sequence of the precursor comprising the leader sequence is set out in SEQ ID NO. 49.

The amino acid sequence of the mature form of gallidermin is set out in SEQ ID NO. 52, and the amino acid sequence of the precursor comprising the leader sequence is set out in SEQ ID NO. 51.

The amino acid sequence of the mature form of mersacidin is set out in SEQ ID NO. 54 and the amino acid sequence of the precursor comprising the leader sequence is set out in SEQ ID NO. 53.

The amino acid sequence of the mature form of actagardine is set out in SEQ ID NO. 56, and the amino acid sequence of the precursor comprising the leader sequence is set out in SEQ ID NO. 55.

The amino acid sequence of the mature form of cinnamycin is set out in SEQ ID NO. 58, and the amino acid sequence of the precursor comprising the leader sequence is set out in SEQ ID NO. 57.

The amino acid sequence of the mature form of duramycin is set out in SEQ ID NO. 60 and the amino acid sequence of the precursor comprising the leader sequence is set out in SEQ ID NO. 59.

The amino acid sequence of the mature form of ancovenin is set out in SEQ ID NO. 61.

Enterococcal cytolysin is a two-peptide system comprising cytolysin ClyLl and ClyLs. The amino acid sequence of the mature form of cytolysin ClyLl is set out in SEQ ID NO. 62, and the amino acid sequence of the precursor comprising the leader sequence is set out in SEQ ID NO. 63. The amino acid sequence of the mature form of cytolysin ClyLs is set out in SEQ ID NO. 64, and the amino acid sequence of the precursor comprising the leader sequence is set out in SEQ ID NO. 65.

Staphylococcin C55 is also a two-peptide bacteriocin comprising SacbA and SacaA. The amino acid sequence of the precursor of SacbA comprising the leader sequence is set out in SEQ ID NO: 66. The amino acid sequence of the precursor of SacaA comprising the leader sequence is set out in SEQ ID NO: 67.

The amino acid sequence of the mature form of mutacin is set out in SEQ ID NO. 69, and the amino acid sequence of the precursor comprising the leader sequence is set out in SEQ ID NO. 68.

In contrast to Class I, Class II bacteriocins are unmodified peptides. Class II bacteriocins include peptides which are typically 30-60 amino acids in length (less than 10 kDa in size), and exhibit the properties of heat tolerance, positive charge and lack of lanthionine or other modified amino acids. Class II bacteriocins are sub-divided into at least 4 sub-classes, including Class IIA, IIB, IIC, and IID.

Class II bacteriocins include multi-peptide bacteriocins, which may comprise for example 2, 3 or 4 peptides, e.g. garvicin KS (see Ovchinnikov et al., Applied and Environmental Microbiology, 2016, 82 (17), 5216-5224). The component peptides of such bacteriocins may be produced as leaderless peptides.

Class IIA bacteriocins include, but are not limited to, Listereria-active peptides with a consensus amino acid sequence of YGNGGVXaaC (SEQ ID NO. 70, where Xaa is any amino acid) at the N-terminal. In an embodiment the Class IIA bacteriocin is pediocin PA-1.

Class IIB bacteriocins typically require two different unmodified peptides to form a fully active bacteriocin complex, and include for example lactococcin G, plantaricin EF, plantaricin JK, plantaricin NC08, lactacin F, and ABP-118.

Class IIC bacteriocins include circular bacteriocins such as carnocyclin A and enterocin AS-48.

Class IID bacteriocins include linear, non-pediocin-like single-peptide bacteriocins, including lactococcin A, lactoccin B, garvicin Q and epidermin NI01.

The Class II bacteriocin may be selected from any one or more, or any combination, of the sub-classes IIA to IID. In an embodiment the Class II bacteriocin is of sub-class IIA, IIB or IID.

By way of representative example, the following amino acid sequences of Class II bacteriocins may be mentioned.

The amino acid sequence of the mature form of the Class IIA pediocin PA-1 is set out in SEQ ID NO. 3, and the amino acid sequence of the precursor comprising the leader sequence is set out in SEQ ID NO. 2. The pediocin PA-1 leader sequence is as set out in SEQ ID. NO. 4.

The amino acid sequence of the mature form of the α-chain of Class IIB lactococcin G is set out in SEQ ID NO. 10, and the amino acid sequence of the α-chain precursor comprising the leader sequence is set out in SEQ ID NO. 9. The amino acid sequence of the mature form of the β-chain of lactococcin G is set out in SEQ ID NO. 11, and the amino acid sequence of the β-chain precursor comprising the leader sequence is set out in SEQ ID NO. 12.

The amino acid sequence of the mature form of the E-chain of Class IIB plantaricin EF is set out in SEQ ID NO. 14, and the amino acid sequence of the E-chain precursor comprising the leader sequence is set out in SEQ ID NO. 13. The amino acid sequence of the mature form of the F-chain of plantaricin EF is set out in SEQ ID NO. 16, and the amino acid sequence of the β-chain precursor comprising the leader sequence is set out in SEQ ID NO. 15.

The amino acid sequence of the mature form of the J-chain of Class IIB planataricin JK is set out in SEQ ID NO.18, and the amino acid sequence of the J-chain precursor comprising the leader sequence is set out in SEQ ID NO.17. The amino acid sequence of the mature form of the K-chain of plantaricin JK is set out in SEQ ID NO. 20, and the amino acid sequence of the K-chain precursor comprising the leader sequence is set out in SEQ ID NO. 19.

The amino acid sequence of the mature form of the α-chain of Class IIB plantaricin NC08 is set out in SEQ ID NO. 22, and the amino acid sequence of the α-chain precursor comprising the leader sequence is set out in SEQ ID NO.21. The amino acid sequence of the mature form of the β-chain of plantaricin NC08 is set out in SEQ ID NO. 24, and the amino acid sequence of the β-chain precursor comprising the leader sequence is set out in SEQ ID NO. 23.

The amino acid sequence of the mature form of Class IID lactococcin A is set out in SEQ ID NO. 26, and the amino acid sequence of the precursor comprising the leader sequence is set out in SEQ ID NO. 25.

The amino acid sequence of the mature form of Class IID lactococcin B is set out in SEQ ID NO. 28, and the amino acid sequence of the precursor comprising the leader sequence is set out in SEQ ID NO. 27.

The amino acid sequence of the mature form of Class IID garvicin Q is set out in SEQ ID NO. 30, and the amino acid sequence of the precursor comprising the leader sequence is set out in SEQ ID NO. 29.

Any of these class I or class II bacteriocins, or variants thereof, or indeed more broadly any Class I or II bacteriocin known in the art may be expressed and produced according to the methods herein.

As examples of Gram-negative bacteriocins (that is bacteriocins of Gram-negative bacteria), mention may be made of microcins, colicins, and colicin-like bacteriocins. Such bacteriocins are described for example in Yang et al., Frontiers in Microbiology, May 2014, volume 5, Article 241, and their sequences and those of their biosynthetic genes may be retrieved from the literature and publically available databases.

In the methods herein, a bacteriocin polypeptide is expressed. As noted above, bacteriocins may be expressed as inactive precursors which are activated by proteolytic cleavage to remove the leader sequence and release the mature active form. Some bacteriocins are single-peptide products and others are multi-peptide products, which comprise two or more peptide chains to form a bacteriocin complex. The individual component polypeptides of such a bacteriocin complex are not in themselves fully active on their own, but may exhibit some activity. The term “bacteriocin polypeptide” as used herein includes mature bacteriocin polypeptides, precursor polypeptides and component polypeptides of multi-peptide bacteriocin polypeptides. Thus, an individual or particular bacteriocin polypeptide may be active, partially active, or inactive, depending on its form. Further, certain bacteriocins are post-translationally modified. The term “bacteriocin polypeptide” includes modified and unmodified polypeptides, that is the polypeptides that are the initial or direct translation products and polypeptides that have been post-translationally modified. Further, the terms “polypeptide” and “peptide” are used synonymously and interchangeably herein, and are not limited by size or length of the peptide chain.

Depending on the bacteriocin to be produced, in order to avoid toxicity of the bacteriocin to the coryneform bacterial host cells, in some embodiments the bacteriocin polypeptide which is expressed and produced by the cell may be selected to be produced in inactive form, or in a partially active form, i.e. having reduced activity, or minimal activity, such that it may be tolerated by the production host cells. The inactive form may be an inactive precursor, or the inactive or partially active form may be a single polypeptide of a multi-peptide bacteriocin, or the inactive or partially active form may be produced as fewer component polypeptides (i.e. chains) than are required for activity, or for full activity. This is described further below. In other embodiments, the bacteriocin may be produced in active form, whether as a multi-peptide complex, or as a mature bacteriocin. In such cases, the coryneform bacterial host may be resistant to (i.e. not susceptible to) the bacteriocin that is produced.

The bacteriocin polypeptide may be a native bacteriocin polypeptide, that is a polypeptide as occurs in nature, or it may be a variant of a native polypeptide. A variant may comprise an amino acid sequence, which is sequence-modified with respect to the native, or wild-type amino acid sequence, e.g. with respect to a reference amino acid sequence as given above, or more generally with respect to the native sequence of the bacteriocin. The sequence modifications may comprise one or more amino acid substitutions, deletions or additions, e.g. 2, 3, 4, 5 or 6 or more amino acid sequence modifications. An amino acid substitution may be a conservative substitution. The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including amino acids with basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus, a conservative amino acid substitution may be considered to be a substitution in which a particular amino acid residue is substituted for a different amino acid residue in the same family.

Thus, the bacteriocin may have an amino acid sequence as set out in any one of the above-mentioned SEQ ID NOs or an amino acid sequence having at least 80% sequence identity thereto, more particularly at least 85, 90, 90, 91, 92, 93, 94, 95, 96, 97, 98 or 99% sequence identity thereto. In certain embodiments the SEQ ID NO. may be the SEQ ID NO. representing the mature sequence of the bacteriocin as set out above. In other embodiments, the SEQ ID NO. may be the SEQ ID NO. representing the precursor polypeptide sequence set out above, which comprises the leader, or “pre” sequence.

Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet., 16, (6) pp276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32(5):1792-1797, 2004) may be used for multiple sequence alignments, though any other appropriate programme may be used. Another suitable alignment programme is BLAST, using the blastp algorithm for protein alignments and the blastn algorithm for nucleic acid alignments. Whether the alignment is pairwise or multiple, it is performed globally (i.e. across the entirety of the reference sequence) rather than locally. Thus, references to % sequence identity herein are with respect to the entire sequence of the stated, referenced, SEQ ID NO.

Sequence alignments and % identity calculations may be determined using for instance standard Clustal Omega parameters: matrix Gonnet, gap opening penalty 6, gap extension penalty 1. Alternatively, the standard EMBOSS Needle parameters may be used: matrix BLOSUM62, gap opening penalty 10, gap extension penalty 0.5. Any other suitable parameters may alternatively be used.

The skilled person will understand that leader sequences may be varied, and indeed leader sequences from different polypeptides may be substituted for the native leader of the bacteriocin polypeptide, including leaders from different bacteriocins. The bacteriocin polypeptide may accordingly be a chimeric bacteriocin comprising a heterologous leader sequence linked to the mature bacteriocin sequence. By heterologous in this context is meant that the leader is not the leader which occurs naturally with the bacteriocin. The leader in the chimeric bacteriocin polypeptide is thus a non-native leader, in the sense that it is not native to the bacteriocin in question. The leader sequence may be obtained or derived from a different polypeptide, e.g. a different bacteriocin. Production systems based on providing chimeric bacteriocin polypeptides with particular leader sequences for use with particular transport and export are discussed in more detail further below.

A variant polypeptide retains the activity or function of the polypeptide from which it is derived, or obtained (i.e. of the parent polypeptide, or in other words the source or “original” polypeptide). As noted above, a bacteriocin polypeptide as expressed or produced may not be active, or fully active, on it its own, but may contribute to bacteriocin activity in the context of a complex of which it is a constituent part, and/or may become activated by proteolytic processing, e.g. by cleavage to remove a leader sequence. Accordingly, “activity” of “function” is to be understood with this in mind. Thus, the variant polypeptide may perform the role of the polypeptide from which it is derived or obtained. In the context of bacteriocin polypeptides, the variant retains the bacteriocin activity of the mature or active bacteriocin polypeptide from which it is derived, or the ability to contribute to bacteriocin activity as part of a bacteriocin complex, or the latent bacteriocin activity of a precursor polypeptide. In particular, a variant polypeptide may retain at least 50, 60, 70, 75, 80, 85, 90 or 95% of the activity or latent activity of the polypeptide from which it is derived or obtained.

In terms of bacteriocin activity, this may be assessed by determining inhibition of growth of an indicator or test organism which is susceptible to the bacteriocin in question. Methods and procedures for determining antimicrobial activity by growth inhibition tests in the presence of the bacteriocin on solid or liquid media are well known in the art.

The coryneform bacterial host is modified to produce the bacteriocin polypeptide by introducing a heterologous nucleic acid molecule, which encodes the bacteriocin polypeptide. By “modified” is meant that the bacteria are subject to genetic modification, or genetic engineering. Modified bacteria are non-native bacteria, that is they do not occur in nature. In other words, they are recombinant, or have been recombinantly modified. In particular, a modified bacterial cell is an engineered bacterial cell which comprises a heterologous nucleic acid molecule. By “heterologous” is meant that the nucleic acid molecule is non-native to the coryneform bacteria, that is, it does not occur in nature in the bacteria. In particular, the nucleic acid molecule comprises a nucleotide sequence which encodes the bacteriocin polypeptide and which is heterologous to the bacteria. The bacteria are thus modified to produce a bacteriocin, or a bacteriocin polypeptide (e.g. a precursor or component polypeptide) which is heterologous to the bacteria. In an embodiment, the bacteria are transformed bacteria, but any means of introducing the nucleic acid molecule into the bacteria is encompassed.

As noted above, the nucleic acid molecule which is introduced to modify the bacteria comprises a nucleotide sequence which encodes the bacteriocin polypeptide. The term “nucleotide sequence” is used herein synonymously and interchangeably with “gene” or “gene sequence” to refer to a sequence encoding the polypeptide in question. In particular, the use of the term “gene” herein does not imply or require the presence with the coding sequence of any promoter sequence or other expression control sequence. Thus, the term “gene” does not imply or require that the native promoter or other control sequence of the native gene is present, merely a coding sequence encoding the stated polypeptide.

The nucleotide sequence, or gene, encoding the bacteriocin polypeptide may be regarded as representing a structural gene for the bacteriocin polypeptide. In the case of bacteriocin polypeptides which are post-translationally modified, the structural gene encodes the polypeptide which is subjected to the post-translational modification to produce the bacteriocin produce. The structural gene thus encodes the initial bacteriocin translation product, or basic or backbone polypeptide which is subsequently modified. The structural gene encodes the structural polypeptide which forms the bacteriocin.

As will be discussed in more detail below, the production machinery of a native producer of a bacteriocin is typically encoded by a gene cluster, which may comprise one or more operons (an operon being a group of genes under common regulatory control, e.g. under the control of the same promoter). The genes in the gene cluster or operon may comprise, in addition to the structural gene, genes coding for one or more transport and export proteins (e.g. a transporter protein). A gene cluster or operon may also typically include a protease for cleavage of the leader, for secretion of a mature bacteriocin or bacteriocin polypeptide. In some cases, the gene cluster or operon may include or more genes encoding a protein which confers or provides the bacteria with immunity to the bacteriocin. Such a protein is referred to herein as an “immunity protein” and includes structural proteins, such as a lipoprotein, enzymes, or transporter proteins. The gene cluster or operon may also include one or more genes involved in the biosynthesis of the bacteriocin. For example, where the bacteriocin polypeptide encoded by the structural gene is subject to modification, the gene cluster or operon may include one or more proteins, e.g. enzymes, involved with chemical modification, e.g. cyclase enzymes, or dehydratase enzymes, or enzymes involved with addition or modification of chemical groups, e.g. in the formation of lanthionine or other unusual amino acids. Such biosynthetic and modification enzymes and other proteins involved in the biosynthesis or modification and/or transport or export of the bacteriocin or bacteriocin polypeptide are collectively referred to herein as “processing and/or transport proteins”. Such proteins include dedicated proteases which cleave leader sequences and transporter proteins. Transporter proteins may be involved in both transport of the polypeptide and cleavage of the leader, and in some cases more than one protein may be required for transport and cleavage.

Depending on the bacteriocin, and the strategy chosen for the production of the bacteriocin or bacteriocin polypeptide, the bacteria may be modified to express one or more other genes from the gene cluster for the bacteriocin in question. These may include processing proteins for biosynthesis or modification of the bacteriocin where appropriate, and/or transport proteins (e.g. transporters), and/or immunity genes. In some cases, a protease-encoding gene may be included, for proteolytic cleavage of a precursor bacteriocin polypeptide. In other cases, where it is desirable to express and recover the bacteriocin polypeptide as a precursor, the protease-encoding gene from the operon may be omitted. In some embodiments it may be desirable to include one or more genes encoding transport and/or export proteins from the gene cluster. In other embodiments, the endogenous transport and/or export machinery of the host coryneform bacteria may be used. In still other embodiments genes encoding transport and/or export machinery may be introduced which are heterologous to both the bacteriocin and the host.

Thus, at its most basic, only the structural gene is required to be introduced into the coryneform bacterial host. However, other genes may optionally be included, e.g. one or more genes encoding (a) processing protein(s), and/or one or more genes encoding (a) transport protein(s), and/or one or more genes encoding (an) immunity protein(s). The additional genes may be provided in the same nucleic acid molecule as the structural gene encoding the bacteriocin polypeptide, or in one or more different, or separate, nucleic acid molecules. The various genes may be expressed co-ordinately with the structural genes, e.g. under the same regulatory control, or under separate regulatory control.

In one embodiment, the nucleic acid molecule which is introduced comprises a synthetic operon comprising:

(i) promoter controlling the expression of the following genes;

(ii) a structural gene encoding the bacteriocin polypeptide;

(iii) optionally, one or more genes encoding processing and/or transport proteins for production of said bacteriocin polypeptide; and/or

(iv) optionally, one or more genes which provide the bacterial strain with immunity to the bacteriocin.

As discussed above, the structural gene may comprise a heterologous leader sequence. In this embodiment, the synthetic operon comprises:

(i) a promoter controlling the expression of the following genes;

(ii) a structural gene encoding the bacteriocin polypeptide which is a chimeric bacteriocin polypeptide which comprises a heterologous leader sequence, which is the leader sequence of a second bacteriocin;

(iii) one or more genes encoding processing and/or transport proteins for production of said bacteriocin polypeptide wherein said genes are processing and/or transport proteins for processing and/or transporting said second bacteriocin; and

(iv) optionally, one or more genes which provide the bacterial strain with immunity to the bacteriocin.

The chimeric bacteriocin polypeptide also contains the sequence of the mature bacteriocin polypeptide to be expressed. An example of this embodiment is shown in FIGS. 13B and E for class II and I bacteriocin expression, respectively and as in Example 6 for the production of flavucin. In these cases the second bacteriocin whose expression and processing machinery is used is pediocin or nisin, respectively.

The heterologous leader sequence may also be provided by a sequence that is recognized by the Sec pathway of the bacterial strain to achieve export of the bacteriocin polypeptide by the endogenous export Sec pathway. This leader sequence is referred to herein as the Sec-dependent leader sequence. Such a sequence provides a peptide which is typically 20 amino acids in length with 3 regions; a positively charged amino terminal, a hydrophobic core and a polar carboxyl-terminal. Any appropriate Sec-dependent leader sequence that is appropriate for the bacterial strain may be used. The Examples illustrate the use of the Sec-dependent secretion signal of aminopeptidase YwaD of Bacillus subtilis.

Thus is a further aspect, the present invention provides a method of the invention in which the synthetic operon comprises:

(i) a promoter controlling the expression of the following genes;

(ii) a structural gene encoding the bacteriocin polypeptide which is a chimeric bacteriocin polypeptide which comprises a heterologous leader sequence which is a Sec-dependent leader sequence; and

(iii) optionally, one or more genes which provide the bacterial strain with immunity to the bacteriocin. By way of example the Sec-dependent leader sequence may be as set forth in SEQ ID NO:79 or an amino acid sequence with at least 80% sequence identity thereto. The construction and introduction (i.e. transfer) of the nucleic acid molecules into the bacteria is described in more detail below.

As noted above, in certain embodiments the coryneform bacteria are not susceptible to the bacteriocin that is produced. In other words, the bacteria are resistant to the bacteriocin. The bacteria may be naturally resistant, or they may be modified to be resistant.

Lack of susceptibility of bacteria to a bacteriocin may arise due to the absence in the bacteria of a receptor, or a target protein, for the bacteriocin in question. This may occur naturally, or the bacterial may be modified to reduce or prevent expression of the receptor or target protein, e.g. to delete or inactivate one or more genes encoding proteins that may act as receptors, or targets, for the bacteriocin.

In many cases, the receptor for a bacteriocin is a transporter protein. For example, the receptors for bacteriocins of the pediocin family on target organisms are the IIC and IID subunits of a family of mannose-specific phosphotransferase systems (PTS^{Man}; PMID: 19477899). In silico analysis of the genome of various strains of C. glutamicum indicated that it did not encode a PTS^{Man}, suggesting it may be resistant against pediocin and other Class II bacteriocins which use this receptor. This has been confirmed by experiments, described in the examples below, which show that C. glutamicum is able to grow in the presence of pediocin. We have thus established that C. glutamicum is a suitable host for production of pediocin, and other Class II bacteriocins, due to a natural resistance to them.

Accordingly, in an embodiment, the bacterial strain does not express a mannose-specific phosphotransferase, and in particular a group I PTS^{Man}. In such an embodiment the bacteriocin may particularly be a Class II bacteriocin, more particularly Class IIA or IID bacteriocin.

Other bacteriocins may use different receptors which may also be absent in strains or species of coryneform bacteria. For example, the maltose ABC transporter has been reported as a receptor for garvicin ML in Lactoococcus lactis (Gabrielssen et al., Antimicrobial Agents and Chemotherapy, 56.6 (2012): 2908-2915). The receptor for lactococcin G in its indicator organism is believed to be undecaprenyl pyrophosphate phosphatase. The receptor for plantaricin EF in its indicator organism is believed to be the membrane-bound magnesium/cobalt efflux protein CorC. The receptor for plantaricin JK in its indicator organism is believed to be an amino acid-polyamine organocation (APC) transporter. The receptor for lactococcin A in its indicator organism is believed to be ManPTSA, and the receptor for lactococcin B and garvicin Q in their indicator organisms is believed to be PTS^{Man}. Such receptors may be naturally absent in strains of coryneform bacteria to be used as production hosts herein, or they may be deleted or inactivated. Techniques for modification of bacteria to delete or inactivate such receptors are well known in the art.

Thus, strains of C. glutamicum or other coryneform bacteria may be obtained or identified in which receptors for other bacteriocins are absent. Furthermore, bacteriocins may use substrate uptake systems as targets or receptors which are not essential to the bacteria, and these may be deleted, to render the bacteria not susceptible to the bacteriocin. Thus, for example, ABC transport systems for maltose (see Henrich et al., 2013, Journal of bacteriology, 195(11), 2573-2584), or indeed the mannose phosphotransferase where it is present, may be deleted or inactivated in the host bacterial strain. Non-essential genes, including membrane transporters which may act as receptors for bacteriocins, are listed in Unthan et al., Biotechnology Journal 10.2 (2015): 290-301.

Alternatively, or additionally, the bacterial strain may be modified to express an immunity protein to the bacteriocin in question. For example, expression of an immunity protein in addition to the absence of a receptor for the bacteriocin may increase resistance of the host bacterial strain to higher levels of bacteriocin production. For example, this may be useful where the bacteriocin is able to use more than one receptor in the host bacteria.

For example, in the case of the Class I bacteriocin nisin, the gene nisi in the nisin gene cluster is believed to encode an immunity protein. Such a gene may be introduced into the host bacterial strain. Where the bacteriocin is a Class I bacteriocin, e.g. a lantibiotic, the host producer will need to be modified to express additional biosynthetic genes, beyond the structural gene, e.g. genes encoding modification enzymes. However, as noted above, not all the genes of a native bacteriocin gene cluster will be required. We have determined that C. glutamicum is sensitive to Class I bacteriocins. Accordingly, an advantageous strategy for production of a Class I bacteriocin in a coryneform bacterial host strain is to produce the bacteriocin as an inactive precursor. Thus, the bacteriocin may be produced by the host bacteria as a precursor polypeptide comprising a leader sequence, i.e. as a “pre-bacteriocin”.

An exemplary Class I bacteriocin is the lantibiotic nisin. As noted above, nisin may occur in a number of variant forms which differ slightly in amino acid sequence. The organization of the nisin gene cluster is described in Ra et al., Microbiology (1996), 142, 1281-1288. In the case of the gene cluster from the producer Lactococcus lactis, this comprises the genes nis(A/Z)BTCIPRKFEG. nis(A/Z)BTCIPRK and nisFEG are comprised in separate operons. nis A/Z is the structural gene for nisin A or nisin Z precursor peptides, respectively. The nisB and nisC are genes for processing enzymes (a dehydratase for modification and a cyclase for formation of the thiolactone rings, respectively). NisT is a dedicated ABC transporter for the modified pre-peptide. Genes nisA/Z, nisB, nisC and nisT represent the minimum gene set necessary from the cluster to be introduced to allow for synthesis of nisin pre-peptide (pre-nisin) by the modified strain. nisP encodes a protease of the leader to form mature and active nisin. This is omitted from the genes that are introduced. As noted above, nisi encodes an immunity protein. This is not necessary but may optionally be included. nisR and nisK encode a 2-component regulatory system for regulation of nisin synthesis. These are not necessary to be included for transfer to the modified host bacteria, since the necessary genes for nisin synthesis can be expressed using regulatory sequences in the systems used to achieve expression in the modified bacteria (e.g. promoters included in expression vectors comprising the nucleic acid molecule which is in introduced). As described in the Examples below, nisRK may conveniently be used in a sensor system for nisin production, since they control expression from the Pₙᵢₛ promoter in response to nisin. They may thus be coupled to a reporter gene, e.g. encoding a reporter protein such as a fluorescent protein. nisFEG encode an ABC transport system which may be involved in nisin self-protection.

Therefore, specific embodiments of the invention, provide a method of the invention for expression of Class I bacteriocins, wherein the nucleic acid molecule comprises a synthetic operon comprising:

(i) a promoter;

(ii) a structural gene encoding the bacteriocin; and

(iii) nisB, nisC and nisT genes, wherein preferably the bacteriocin is nisin. An Example of this method is set forth in Example 2.

Whilst a nucleic acid molecule comprising nucleotide sequences corresponding to or derived from nis(A/Z)BCT may be introduced to produce nisin A or Z, it will be understood that analogous nucleic acid molecules may be prepared comprising nucleotide sequences corresponding to or derived from genes clusters for other lantibiotics, or Class I bacteriocins more generally. The sequences for such gene clusters may be available from publicly available databases or may be obtained by genome sequencing of producer organisms. As noted above, the nucleotide sequences may be codon-optimised for expression in coryneform bacteria, although this is not strictly necessary. Codon optimised sequences for nisZ, nisB, nisT, and nisC are provided in SEQ ID NOs: 71 to 74, respectively.

Further, it has been shown the modification machinery of one lantibiotic, for example nisin, is able to modify other lantibiotics. Accordingly, for production of other lantibiotics, such as those listed above, a nucleotide sequence corresponding to or derived from the structural gene for the lantibiotic may be used in conjunction with nucleotide sequences corresponding to, or derived from, nisin genes, e.g. nisBCT. Therefore, in an alternative embodiment of the invention, there is provided a method of the invention for expression of Class I bacteriocins, wherein the nucleic acid molecule comprises a synthetic operon comprising:

a promoter;

(ii) a structural gene encoding the bacteriocin polypeptide to be expressed which is a chimeric bacteriocin polypeptide which comprises the leader sequence of nisin (or an amino acid sequence with at least 80% sequence identity thereto);

(iii) nisB, nisC and nisT genes, wherein preferably the flavulin. The nisin leader sequence is shown (as part of) SEQ ID NO. 31. An Example of this method is set forth in Example 6 as shown in FIG. 13E.

The strategy of producing the bacteriocin as an inactive precursor may also be employed for the production of a Class II bacteriocin if necessary or desirable, for example where the host strain is susceptible to the bacteriocin.

When the bacteriocin is produced by the bacteria as an inactive precursor polypeptide, it may be recovered, or harvested as such.

An inactive precursor may be processed, e.g. by cleavage to remove the leader sequence to generate a mature bacteriocin. Thus, the method may comprise an additional step of cleaving the inactive precursor.

Conveniently, this may be performed in vitro. Hence, the method may comprise a downstream in vitro processing step to produce a mature, active bacteriocin. For example, the inactive precursor may be harvested, and then subjected to an in vitro cleavage step. The cleavage step involves contacting the precursor with a protease, or proteolytic enzyme, which is capable of cleaving the leader from the precursor. The precursor may be harvested as culture supernatant which may then be contacted, or treated, with the protease. Alternatively, the precursor may be isolated or purified before being contacted, or treated, with the protease, i.e. there may be one or more separation steps before the precursor is subjected to cleavage. In still another embodiment, the growth culture containing the precursor and the host cells may be subjected to the cleavage step directly, before the mature bacteriocin is recovered.

Any suitable protease may be used for this step. It may be the dedicated protease for the bacteriocin in question, or a protease used for cleavage of a different bacteriocin. It may also be a protease such as trypsin, which has been shown to be capable of cleaving the bacteriocin, or proteinase K. Such proteases, including for example protease NisP, may be recombinantly produced for such use (see e.g. Montalbán-López, et al., (2018) Frontiers in microbiology, 9, 160).

Alternatively, the cleavage step may be performed in vivo. Thus, the precursor may be contacted (i.e. may interact) with the protease in vivo, within the host cells, or during transport or export of the bacteriocin polypeptide. This may be achieved in various ways in order to avoid toxicity of the bacteriocin to the host bacterial cells. For example, a protease for cleavage of the precursor may be separately provided to the modified strain, under control of an inducible promoter, and may be induced once the culture step for expression of the bacteriocin polypeptide has been concluded, and the bacteriocin polypeptide has been produced by the bacteria.

In other embodiments, where the bacteria are naturally resistant or have been modified to be resistant to the bacteriocin in question, proteolytic cleavage may take place in vivo. This may be by endogenous proteolytic enzymes present in the host or by introduction of a protease gene to the modified bacteria, e.g. the dedicated protease for the bacteriocin, for example nisP for nisin or other lantibiotics.

In the case of Class II bacteriocins, it is believed that in many cases it will be possible to identify and use a coryneform bacterial host which is resistant to the bacteriocin in question.

An exemplary Class II bacteriocin is pediocin, and in particular pediocin PA-1. The pediocin operon is described in Marugg et al., Appl. Environm. Microbio. 58, 2360-2367 (1992) and Venema et al., Mol. Microbiol. 17, 515-522 (1995). The operon comprised 4 genes. PedA is the structural gene for the precursor, pedB encodes an immunity protein, pedC and pedD encode proteins involved in transport and processing of the precursor to generate and release the mature bacteriocin. The PedB immunity protein confers resistance by a mechanism that depends on the receptor PTS^{man} and therefore this protein is dispensable in coryneform bacterial hosts which lack PTS^{Man}, such as various strains of C. glutamicum including strain CR099.

Thus, a system for expression of pediocin may be constructed based on a nucleotide sequences, which correspond to or are derived from pedA, pedC and pedD. It will be understood that analogous nucleic acid molecules may be prepared comprising nucleotide sequences corresponding to or derived from genes clusters for other Class II bacteriocins. The sequences for such gene clusters may be available from publicly available databases or may be obtained by genome sequencing of producer organisms. As noted above, the nucleotide sequences may be codon-optimised for expression in coryneform bacteria, although this is not strictly necessary.

By way of example, variants of the native gene sequences for pedA, pedC and pedD which have been codon-optimised for expression in C. glutamicum are shown in SEQ ID Nos. 1, 5 and 7 respectively. SEQ ID NOs 6 and 8 show the respective PedC and PedC translation products.

A synthetic operon may be prepared and included in the nucleic acid molecule which is introduced into the modified strain. The synthetic operon may comprise a promoter, a nucleotide sequence encoding the pediocin precursor polypeptide, and nucleotide sequences encoding proteins corresponding to pedC and pedD gene products.

As noted above, leader sequences may be interchangeable between different bacteriocins. For several Class II bacteriocins, particularly those that include a double glycine (GG) motif in their leader sequence it has been shown that the transport and modification machinery may be promiscuous. There have been a number of reports in the literature showing that various bacteriocins may be produced with heterologous leaders and transport/export proteins. For example, the GG-leader of lactococcin A (class IID) and its dedicated ABC transporter can be used to produce pediocin (class IIA). Fusion of the pediocin leader sequence to the coding sequence of colicin V allowed production of active colicin in a strain harbouring this construct and the genes required for pediocin secretion. Similarly, active divergicin A (class IIA) could be produced by fusion of the GG-leaders of leucocin A (class IIA), lactococcin A (class IID) or colicin V (unclassified). Thus, it is expected that class II bacteriocins, and particularly those that carry a GG motif in their leader sequence, can be produced using the pediocin export apparatus. Potential candidates amongst class II bacteriocins include pediocin, lactococcin G, plantaricin EF, plantaricin JK, plantaricin NC08, lactococcin A, lactococcin B, and garvicin Q.

Thus, one strategy is to express a structural gene for a Class II bacteriocin together with processing and transport protein-encoding genes for a different Class II bacteriocin, particularly, a Class IIA. B or D bacteriocin, and more particularly a Class II bacteriocin as listed above. In this way, a standardised production system may be provided for production of different bacteriocins using the same processing and transport protein encoding genes. For example, such a system may be based on the pedC and pedD genes. Thus, the nucleic acid molecule which is introduced may comprise a nucleotide sequence encoding a Class II bacteriocin precursor polypeptide, and nucleotide sequences encoding proteins corresponding to pedC and pedD gene products. Such a nucleic acid molecule may be provided in the form of a synthetic operon comprising a promoter operably linked to the coding nucleotide sequences.

To enable such a method, a nucleic acid molecule may be provided which comprises the pedC and pedD genes, ready for insertion of a desired bacteriocin precursor coding sequence,

Thus, in a further aspect there is provided a recombinant construct for expression of a bacteriocin polypeptide in coryneform bacteria, said construct comprising:

(i) a promoter for controlling the expression of one or more inserted genes;

(ii) an insertion site for insertion of a structural gene encoding a bacteriocin polypeptide, optionally together with one or more genes encoding processing proteins for production of said bacteriocin polypeptide; and/or which provide the coryneform bacteria with immunity to the bacteriocin;

(iii) pedC and pedD genes (or in other words, a nucleotide sequence comprising a sequence corresponding to pedC and pedD) optionally wherein the nucleotide sequences of said genes are codon-optimised for expression in coryneform bacteria.

Part (iii) may alternatively be defined as a nucleotide sequence which comprises the sequences as set forth in SEQ ID NOs. 5 and 7, or a nucleotide sequence with at least 80% sequence identity with said sequences.

The recombinant construct may be comprised in a vector.

The bacteriocin structural gene may encode the bacteriocin with its native leader sequence. However, in another embodiment the bacteriocin may be a chimeric bacteriocin comprising a heterologous leader sequence. In a particular embodiment the chimeric bacteriocin may comprise a pediocin leader sequence. The pediocin leader sequence is shown in SEQ ID NO. 4.

In other embodiments the nucleic acid molecule may comprise a nucleotide sequence encoding a Class II bacteriocin precursor polypeptide, and nucleotide sequences encoding proteins corresponding to the native transport and export proteins for that bacteriocin. Thus, a nucleic acid molecule may be prepared in a manner analogous to that described for pediocin above, based on the native gene cluster for that bacteriocin.

In still further embodiments, it may not be necessary to provide the modified bacterial host with heterologous export or transport proteins, and to use instead the endogenous export and transport proteins of the host. It is known that several bacteriocins may be secreted naturally using general peptide/protein secretion machineries. Reference may be made in this regard to Cintas et al., Applied and Environmental microbiology, 63(11), 4321-4330, (1997); and McCormick et al., (1996), Applied and Environmental microbiology, 62(11), 4095-4099. It has further been reported that C. glutamicum possesses a functional secretory system for efficient export of heterologous proteins via specific signal peptides, which responds also to heterologous signal peptides (see Lee and Kim, 2018, Frontiers in microbiology, 9, 2523 and Freudl, 2017, Journal of biotechnology, 258, 101-109). Thus, where a bacteriocin does not require any further modification other than export and cleavage, it may be produced in C. glutamicum or other coryneform bacteria, by modifying the bacteria to introduce a nucleic acid molecule comprising a nucleotide sequence encoding the bacteriocin polypeptide precursor. The nucleic acid molecule may comprise solely the nucleotide sequence encoding the bacteriocin precursor, i.e. a single gene representing the structural gene for the bacteriocin, and no further genes may be required.

Therefore, specific embodiments of the invention, provide a method of the invention for expression of Class II bacteriocins, wherein the nucleic acid molecule comprises a synthetic operon comprising:

(i) a promoter;

(ii) a structural gene encoding the bacteriocin;

(iii) garC and garD genes; and

(iv) optionally, one or more genes which provide the bacterial strain with immunity to the bacteriocin, wherein preferably the structural gene encodes garvicin Q, or a chimeric bacteriocin polypeptide which comprises the leader sequence of garvicin Q (or an amino acid sequence with at least 80% sequence identity thereto).

An example of this embodiment is shown in FIG. 13C and in Example 4 for the production of garvicin Q. The garvicin Q leader sequence is shown (as part of) SEQ ID NO. 29.

In an alternative the present invention also provides a method of the invention for expression of Class II bacteriocins, wherein the nucleic acid molecule comprises a synthetic operon comprising:

(i) a promoter;

(ii) a structural gene encoding the bacteriocin polypeptide to be expressed which is a chimeric bacteriocin polypeptide which comprises a heterologous leader sequence which is a Sec-dependent leader sequence; and

(iii) optionally, one or more genes which provide the bacterial strain with immunity to the bacteriocin, wherein preferably the structural gene encodes garvicin Q. The Sec-dependent leader may be as described hereinbeofre

The chimeric bacteriocin polypeptide also contains the sequence of the mature bacteriocin polypeptide to be expressed. An example of this embodiment is shown in FIG. 13D and in Example 5 for the production of garvicin Q.

For bacteriocins which are multi-peptide complexes, the individual bacteriocin polypeptide components may be expressed individually in the same bacterial cell, such that a bacteriocin complex may not be produced, or they may be expressed all together, such that a bacteriocin complex may be produced. Where the host is susceptible to the bacteriocin, less than the complete number of component polypeptides may be produced. In this way toxicity of the bacteriocin to the host may be avoided. However, even where the host is not susceptible, it may be desired or beneficial to express component polypeptides separately. Thus, where the Class II bacteriocin is a multi-peptide bacteriocin comprising 2 or more bacteriocin polypeptides, the method may comprises separately expressing each bacteriocin polypeptide in the bacterial strain, harvesting each bacteriocin polypeptide, and combining the bacteriocin polypeptides to prepare a bacteriocin complex. This may be beneficial in controlling the formation of the complex. Different component polypeptides may be present in the complex in different ratios. For example, lactococcin G is fully active in complexes of α and β peptides in a molar ratio of 7:1 or 8:1 respectively. Separate producer strains may be generated for each component polypeptide, and each polypeptide may be produced and recovered separately, purified and combined to produce an optimised product containing both component polypeptides in molar ratios that ensure optimum activity.

As noted above, the bacteriocin may be a variant of a native bacteriocin molecule and may thus be encoded by a nucleotide sequence which is a variant of a native structural gene sequence. Similarly, any further proteins or peptides encoded herein, e.g. processing and/or transport proteins, may also be variant proteins (as indicated above, a variant protein or peptide may comprise one or more amino acid sequence modifications, as discussed above). A variant protein or peptide may be used which retains the activity of the protein or peptide from which it is derived, as also discussed above. The terms used to denote the various genes discussed above (e.g. pedC, pedD, nisA, nisZ, nisB, nisC, nisT, garQ, garC, garD etc.) as used herein include variants of the native gene sequences.

Accordingly, the encoded protein may comprise an amino acid sequence having at least 80% sequence identity, more particularly at least 85, 90, 90, 91, 92, 93, 94, 95, 96, 97, 98 or 99% sequence identity, to the amino acid sequence of the native protein. By way of representative example, the protein may have at least 80%, more particularly at least 85, 90, 90, 91, 92, 93, 94, 95, 96, 97, 98 or 99% sequence identity to the sequence of PedC or PedD as set out in SEQ ID NO.s 6 or 8 respectively. Similarly, the YwaD signal peptide may have at least 80%, more particularly at least 85, 90, 90, 91, 92, 93, 94, 95, 96, 97, 98 or 99% sequence identity to the sequence as set out in SEQ ID NO. 79. Similarly, the nucleotide sequences encoding a variant protein or peptide may comprise at least at least 80%, more particularly at least 85, 90, 90, 91, 92, 93, 94, 95, 96, 97, 98 or 99% sequence identity to the native nucleotide sequence encoding the native protein or peptide, or to the codon-optimized version thereof. By way of representative example, the nucleotide sequence encoding PedA, PedC or PedD may have at least at least 80%, more particularly at least 85, 90, 90, 91, 92, 93, 94, 95, 96, 97, 98 or 99% sequence identity to the sequence as set out in SEQ ID NO.s 1, 5 and 7 respectively. Similarly, the nucleotide sequence encoding NisZ, NisB, NisT or NisC may have at least 80%, more particularly at least 85, 90, 90, 91, 92, 93, 94, 95, 96, 97, 98 or 99% sequence identity to the sequence as set out in SEQ ID NOs. 71 to 74, respectively. Similarly, the nucleotide sequence encoding GarQ, Gad, GarC or GarD may have at least 80%, more particularly at least 85, 90, 90, 91, 92, 93, 94, 95, 96, 97, 98 or 99% sequence identity to the sequence as set out in SEQ ID NOs. 75 to 78, respectively.

The nucleic acid molecule may be constructed or prepared according to methods well known in the art. In an embodiment the nucleic acid molecule may be provided in the form of a recombinant construct comprising the coding nucleotide sequence(s) operably linked to one or more expression control sequence, for example a promoter, optionally with one or more further regulatory sequences. Each coding sequence may be under the control of a separate expression control sequence, but for convenience, all the coding sequences to be introduced may be under the control of the same expression control sequence(s). For example, as noted above, the recombinant construct may take the form of a synthetic operon comprising a promoter operably linked to the coding sequences. A construct comprising a nucleic acid molecule comprising one or more coding sequences and one or more expression control sequences may be referred to herein as an expression construct.

The nucleic acid molecule or recombinant construct, e.g. comprising a synthetic operon, may be comprised within a vector, e.g. for the purposes of cloning, or for expression, e.g. in an expression vector. As used herein, the term “vector” refers to any genetic element capable of serving as a vehicle of genetic transfer, expression, or replication for an exogenous nucleic acid sequence in a host strain. A vector may exist as a single nucleic acid molecule or as two or more separate nucleic acid molecules. Vectors may be single copy vectors or multicopy vectors when present in a host strain.

A particular vector for use herein is an expression vector. In such a vector, one or more genes can be inserted into the vector molecule, in proper orientation and proximity to expression control elements resident in the expression vector molecule so as to direct expression of one or more proteins when the vector molecule is present in the host strain.

Construction of appropriate expression vectors and other recombinant or genetic modification techniques for practising the method herein are well known in the art (see, e.g., Green and Sambrook, Molecular Cloning, A Laboratory Manual, Cold Spring Harbor Laboratory Press (Cold Spring Harbor, N.Y.) (2012), and Ausubel et al., Short Protocols in Molecular Biology, Current Protocols John Wiley and Sons (New Jersey) (2002).

The vector can be a plasmid, cosmid, phagemid or other phage vector, viral vector, episome, an artificial chromosome, e.g. bacterial artificial chromosome (BAC) or P1 artificial chromosome (PAC), or other polynucleotide construct, and may, for example, include one or more selectable marker genes and appropriate expression control sequences.

Generally, regulatory control sequences are operably linked to the coding nucleic acid sequences, and include constitutive, regulatory and inducible promoters, transcription enhancers, transcription terminators, and the like which are well known in the art. As noted above, the coding nucleic acid sequences can be operably linked to one common expression control sequence or linked to different expression control sequences, such as one inducible promoter and one constitutive promoter.

Suitable promoter sequences for expression in bacteria, and particularly coryneform bacteria, are known in the art and include for example the P_{tac} promoter, P_{lac}, P_{trc}, P_{araBAD}, Pₜₑₜ, P_{git1}, and P_{malE1} promoters. It may be advantageous to use a strong promoter. In particular, a strong inducible promoter may be used. The P_{tac} promoter is induced by IPTG.

The choice of the vector will typically depend on the compatibility of the vector with the host strain into which the vector is to be introduced. The vectors may be linear or closed circular plasmids. The vector may also be an autonomously replicating vector, i.e., a vector which exists as an extrachromosomal entity, the replication of which is independent of chromosomal replication, e.g. a plasmid, an extrachromosomal element, a minichromosome, or an artificial chromosome. The vector may contain any means for assuring self-replication. Alternatively, the vector may be one which, when introduced into the host strain, is integrated into the genome and replicated together with the chromosome(s) into which it has been integrated. Integrative plasmids are known in the art. Furthermore, a single vector or plasmid or two or more vectors or plasmids which together contain the total nucleic acid to be introduced into the genome of the host strain, or a transposon may be used.

The vectors may contain one or more selectable markers which permit easy selection of transformed cells. The selectable marker genes can, for example, encode detectable products, e.g. fluorescent proteins, provide resistance to antibiotics or toxins, complement auxotrophic deficiencies, or supply critical nutrients not in the culture media, and/or provide for control of chromosomal integration. Examples of bacterial selectable markers are markers which confer antibiotic resistance such as ampicillin, kanamycin, chloramphenicol, or tetracycline resistance.

The vectors may also contain one or more elements that permit integration of the vector into the host strain genome or autonomous replication of the vector in the host independent of the genome. For integration into the host strain genome, the vector may rely on an encoding nucleic acid sequence or other element of the vector for integration into the genome by homologous or non-homologous recombination. CRISPR-based systems may also be used to achieve integration. For autonomous replication, the vector may further comprise an origin of replication enabling the vector to replicate autonomously in the strain in question. The origin of replication may be any plasmid replicator mediating autonomous replication which functions in a cell. The term “origin of replication” or “plasmid replicator” is defined herein as a nucleotide sequence that enables a plasmid or vector to replicate in vivo.

The modified strain is typically prepared by introducing, typically via transformation, one or more vectors as described herein, using standard methods known in the art (see, e.g., Sambrook et al., 2012, supra). The introduction of a vector into a bacterial host cell may, for instance, be effected by protoplast transformation (see, e.g. Chang and Cohen, 1979, Molecular General Genetics 168: 111-115), using competent cells (see, e.g., Young and Spizizen, 1961, Journal of Bacteriology 81: 823-829, or Dubnau and Davidoff-Abelson, 1971, Journal of Molecular Biology 56: 209-221), electroporation (see, e.g. Shigekawa and Dower, 1988, Biotechniques 6: 742-751), or conjugation (see, e.g. Koehler and Thome, 1987, Journal of Bacteriology 169: 5771-5278). Other methods include lipofection and particle gun acceleration.

As described above, the vector, once introduced, may be maintained as a chromosomal integrant or as a self-replicating extra-chromosomal vector. The transformation can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. Expression levels can further be optimized to obtain sufficient expression using methods well known in the art.

In an embodiment the expression vector herein is a plasmid, which may be a self-replicating plasmid, or an integrative plasmid.

The precise nature of the plasmid is not critical. Numerous plasmids suitable for use in coryneform bacteria are known in the art. Representative examples of possible plasmids which may be used as the basis for expression vectors for the uses herein include the plasmids designated: pEKEX2, pXMJ19, pPBEx2, pVWEx1, pOGOduet, pRG-dCas9, pCLTON1 or pK19mobsacB. Other examples include the expression vectors pRG_Duet1 and pRG_Duet2 (from Gauttam, et al., (2019) Plasmid, 101, 20-27).

More generally, heterologous expression in strains of coryneform bacteria can be achieved using many plasmids that replicate in the cytoplasm of the bacteria. A wide range of plasmids are described in Eggeling, L., & Bott, M. (Eds.). (2005). Handbook of Corynebacterium glutamicum. CRC press. Furthermore in the literature a wealth of other plasmids have been described which are derivatives of well-known plasmids, or use well known replicons, and any of these may be used e.g. ON1, the pRG-plasmids, or the plasmids described in Bakkes et al (2020) Plasmid, 112, 102540 and Henke et al., Microorganisms 2021, 9, 204.

Once the nucleic acid molecule has been introduced into the host strain, the modified bacteria are grown, or cultured, under conditions suitable for expression of the bacteriocin polypeptide. Once again, procedures and conditions for this are known in the art, and this can readily be achieved according to techniques and principles well known in the art.

Growth media suitable for coryneform bacteria are known in the art and include for example TY medium. However, as mentioned above, one of the advantages of using coryneform bacteria as production host is that a wide range of materials and media can be used as growth substrates, including waste materials or by-products from industrial processes.

Accordingly, a simple carbon source may be used that is derived from a renewable feedstock, including but not limited to, carbohydrates from corn, cane, or other plant materials, natural gas, or lignocellulosic biomass; waste products such as municipal solid waste, glycerol, flu-gas, syn-gas, carbon dioxide; or the carbon streams resulting from the reformation of organic materials such as biomass, natural gas, or other carbonaceous materials.

A “feedstock” is the raw material that is used in the manufacture of a product or for an industrial process. A “renewable feedstock” is a raw material that is derived from renewable materials such as a biological material, e.g., plant matter and that can be replaced through natural means (e.g. corn, cane, lignocellulosic biomass) or waste products such as municipal solid waste, glycerol, free fatty acids, flu-gas, or syn-gas; carbon dioxide, or the like.

Exemplary, simple carbon sources include, but are not limited to monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan;

disaccharides, such as sucrose, maltose, cellobiose, and turanose; cellulosic material and variants such as hemicelluloses, methyl cellulose and sodium carboxymethyl cellulose; saturated or unsaturated fatty acids, succinate, lactate, acetate; alcohols, such as ethanol, methanol, and propanol; glycerol, or mixtures thereof. In one embodiment, the simple carbon source is derived from corn, sugar cane, sorghum, beet, switch grass, ensilage, straw, lumber, pulp, sewage, garbage, cellulosic urban waste, flu-gas, syn-gas, or carbon dioxide. The simple carbon source can also be a product of photosynthesis, such as glucose.

The simple carbon source may be derived from biomass. An exemplary source of biomass is plant matter or vegetation, such as corn, sugar cane, or switchgrass. Another exemplary source of biomass is metabolic waste products, such as animal matter (e.g. cow manure). Further exemplary sources of biomass include algae and other marine plants. Biomass also includes waste products from industry, agriculture, forestry, and households, including, but not limited to, fermentation waste, ensilage, straw, lumber, sewage, garbage, cellulosic urban waste, and food leftovers.

After the bacteriocin polypeptide has been expressed, it may be harvested, or in other words recovered, or collected from the culture.

Advantageously, the bacteriocin polypeptide is produced as a secreted product. That is, it is secreted by the producing host cells into the extracellular medium. A protein that is produced extracellularly, may readily be recovered by separating the extracellular medium, i.e. the supernatant, from the cells. Procedures for this are known in the art and include for example centrifugation or filtration.

Although less preferred, bacteriocin polypeptides which are produced intracellularly may be extracted or separated from bacterial cells by cell lysis procedures, again well known in the art.

At its simplest, the bacteriocin product may be recovered as a supernatant which contains the bacteriocin polypeptide. As referred to herein harvesting the bacteriocin constitutes recovering the produced bacteriocin polypeptides from the cells, e.g. by collecting the supernatant or the cell lysate.

However, the bacteriocin polypeptide may be isolated or separated from the supernatant (or indeed if necessary, from a cell lysate) by protein purification procedures known in the art. These include for example, precipitation, chromatography or filtration methods, e.g. ammonium sulphate precipitation, ion exchange chromatography, reverse phase chromatography, size exclusion chromatography, gel filtration, HPLC methods and such like. Any desired or convenient combination of purification methods may be used.

Thus, the methods herein may comprise a further step of isolating or purifying the bacteriocin polypeptide.

As noted above, the bacteriocin polypeptide may be recovered, and hence may be purified as a mature active bacteriocin, as an inactive precursor, or as a component polypeptide.

In a further step, the obtained bacteriocin polypeptide may be processed into a product. This may be by cleavage to provide a mature peptide or by any other process required to provide an active bacteriocin. Processing may also comprise combining the isolated or purified bacteriocin polypeptide with other components, e.g. reagents to improve stability or activity.

The invention also extends to a product obtainable by a method as defined herein.

The invention will now be described in more detail in the following non-limiting examples with reference to the following figures.

### BRIEF DESCRIPTION OF FIGURES

FIG. 1 shows (A) Microtiter plate growth inhibition assay using L. monocytogenes EGDe::pIMK2 (upper panel) or L. innocua LMG2785::pIMK2 (lower panel) to determine activity of a pediocin PA-1 standard solution at the indicated concentration in the assay. (B) Growth inhibition of L. monocytogenes EGDe::pIMK2 by supernatants of P. acidilactici 347 (Paci) collected in early stationary growth phase at the indicated dilution. Values are mean±standard deviation of at least n=3 independent experiments per condition. Dilution series sterile H₂O (A) or MRS medium (B) were used as negative controls. Hatched bars indicate growth of the indicator strain without addition of diluted supernatant or pediocin, i.e. maximum growth. The bottom and top lines indicate OD₆₀₀ of the positive (i.e. complete inhibition of growth) or negative (i.e. in the absence of an antimicrobial peptide), respectively. The broken middle line represents growth inhibition of 50%, i.e. the threshold to calculate bacteriocin units.

FIG. 2 shows (A) Growth (OD₆₀₀) of C. glutamicum CR099 in 96-well microtiter plates in the presence of pediocin (black bars) nisin (grey), or bacitracin at the indicated concentrations. Values are mean±standard deviation of n=4-7 independent experiments. (B) Growth of C. glutamicum CR099 in standard batch culture in 2xTY medium with 2% Glc in the presence (+PA-1) or absence (−PA-1) of 2.0 μg/mL pediocin. Values are mean±standard deviation of n=4 independent experiments. Growth rates were calculated during exponential growth phase. (C) Growth inhibition of L. monocytogenes EGDe::pIMK2 by supernatants of C. glutamicum CR099 in 2xTY medium with 2% Glc plus 2 μg/mL pure pediocin (+CR099). Supernatants were collected during the experiment shown in (B) at the indicated time-points. As controls, pediocin was incubated under the same conditions in sterile medium (−CR099). Values are OD₆₀₀ of the indicator strain L. monocytogenes EGDe in a growth assay at the indicated dilutions of the samples and are mean±standard deviation of n=3 independent experiments. In all graphs, the bottom and top lines indicate OD₆₀₀ of the positive (i.e. complete inhibition of growth) or negative (i.e. in the absence of an antimicrobial peptide), respectively. The broken middle lines represent growth inhibition of 50%, i.e. the threshold to calculate bacteriocin units.

FIG. 3 shows (A) Genetic organization of the operon of P. acidilactici PAC1.0 for biosynthesis of pediocin PA-1. (B) Plasmid map of pEKEx2-pedACD introduced into C. glutamicum CR099. (C) Growth inhibition of L. monocytogenes EGDe::pIMK2 by supernatants of C. glutamicum CR099/pEKEx2-pedACD (pedACD) or the empty vector control strain C. glutamicum CR099/pEKEx2 (ped0). Bacteria were grown in 5 ml 2xTY medium in glass tubes and 2h after inoculation Glc (2 w/v) and IPTG (0.1 mM) were added to induce production of pediocin. Activity was measured in 2-fold serial dilutions of culture supernatants after o/N growth, i.e. early stationary growth phase, using the growth assay with L. monocytogenes EGDe::pIMK2 as indicator. Values are OD₆₀₀ of the indicator strain and are mean±standard deviation of n=9 independent experiments. Hatched bars indicate growth of the indicator strain without addition of diluted supernatant or pediocin, i.e. maximum growth. The bottom and top lines indicate OD₆₀₀ of the positive (i.e. complete inhibition of growth) or negative (i.e. in the absence of an antimicrobial peptide), respectively. The broken middle lines represent growth inhibition of 50%, i.e. the threshold to calculate bacteriocin units.

FIG. 4 shows (A) Growth of C. glutamicum CR099/pEKEx-ped0 (ped0) and C. glutamicum CR099/pEKEx-pedACD (pedACD) in 2xTY medium in baffled (+) and non-baffled (−) Erlenmeyer flasks. 2h after inoculation Glc (2% w/v) and IPTG (0.1 mM) were added to induce production of pediocin (B) Growth inhibition of L. innocua LMG2785::pIMK2 by different dilutions of supernatants of C. glutamicum CR099/pEKEx2-pedACD (pedACD) collected at the indicated timepoints during the growth experiment shown in (A). All values are mean±standard deviation of n=3 independent experiments. The bottom and top lines indicate OD₆₀₀ of the positive (i.e. complete inhibition of growth) or negative (i.e. in the absence of an antimicrobial peptide), respectively. The broken middle lines represent growth inhibition of 50%, i.e. the threshold to calculate bacteriocin units.

FIG. 5 shows ion exchange chromatography of ammonium sulphate precipitated supernatants of P. acidilactici 347 (left panel) or C. glutamicum CR099/pEKEx-pedACD (right panel) following growth in non-baffled (−) Erlenmeyer flasks in 2xTY medium with glucose (2% w/v) and IPTG (0.1 mM). Black lines indicate absorbance at 280 nm in milli arbitrary units (mAU) and grey lines conductivity in mS/cm. Broken vertical lines indicate the boundaries of the peak fractions of the eluate collected for further analysis.

FIG. 6 shows (A) Growth inhibitory activity in different dilutions of fractions F8-10 of ion exchange chromatography of precipitated supernatant of C. glutamicum CR099/pEKEx-pedACD grow in 2xTY medium with 2% Glc in non-baffled Erlenmeyer flasks overnight. Samples were analyzed untreated or following incubation with proteinase K (+protK). All values are mean±standard deviation of n=3 independent experiments. The bottom and top lines indicate OD₆₀₀ at baseline (i.e. complete inhibition of growth) or in the absence of antimicrobial peptides, respectively. The broken middle line represents growth inhibition of 50%, i.e. the threshold to calculate bacteriocin units. (B) SDS-PAGE of peak fractions F9 (IEX F9) of supernatants of C. glutamicum CR099/pEKEx-pedACD (CR099 pedACD) or P. acidilactici 347 (P. aci) analyzed by ion exchange chromatography. Pure pediocin (PA-1) was analyzed on the same gel as control. (C) Size exclusion chromatography of pooled ion exchange fractions F8-F10 from supernatants of P. acidilactici 347 (P. aci, middle panel) or C. glutamicum CR099/pEKEx-pedACD (Cg pedACD; lower panel) or pure pediocin (PA-1) as a control. The box indicates a signal in all samples with a size corresponding to pure pediocin. (D) Mass spectrometry of active fractions after reverse phase chromatography of pediocin PA-1 (upper panel) and pooled active cation-exchange fractions from supernatants C. glutamicum CR099/pEKEx-pedACD (lower panel) before applying to reverse phase chromatography. The boxes highlight peaks observed in both samples with almost identical mass/charge ratio (m/z) that match the calculated m/z of pediocin PA-1.

FIG. 7 shows growth inhibition of L. monocytogenes EGDe by different dilutions of supernatants of C. glutamicum CR099 strains harboring pEKEx2-derived constructs with ped genes in different combinations (A) or in different order and copy number (B). Bacteria were grown in 5 mL 2xTY medium in glass tubes and 2h after inoculation Glc (2% w/v) and IPTG (0.1 mM) were added. All values are mean OD₆₀₀±standard deviation of n=3 independent experiments. The bottom and top lines indicate the OD₆₀₀ at baseline (i.e. complete inhibition of growth) or in the absence of antimicrobial peptides, respectively. The broken middle lines represent growth inhibition of 50%, i.e. the threshold to calculate bacteriocin units.

FIG. 8 shows (A) Offline measurements of substrate, biomass and pediocin activity and (B) online measurements of critical process parameters during fermentation of C. glutamicum CR099/pEKEx-pedACD grow a bioreactor in CGXII medium supplemented with 2% Glc, 16 g/L tryptone and 10 g/L yeast extract under controlled fed-batch conditions (in A: biomass (DCW), bacteriocin acitivity (activity), glucose (Glc) and glutamate (Glu); in B: Dissolved oxygen (DO₂), agitator speed (agitation) and aeration rate (aeration)). The timepoint of induction (t=7.5 h) is indicated by the vertical line.

FIG. 9 shows ion exchange chromatography of ammonium sulphate precipitated supernatants of C. glutamicum CR099/pEKEx-nisZBTC^{opt} CR099/pPBEx-nisZBTC^{opt} and CR099/pXMJ-nisZBTC^{opt} grown in 2xTY medium with glucose (2% w/v) and IPTG (0.1 mM). Black lines indicate absorbance at 214 nm in milli arbitrary units (mAU) and grey lines conductivity in mS/cm. Broken lines indicate the boundaries of the peak fractions.

FIG. 10 shows (A) Growth inhibition of C. glutamicum CR099 by nisin standard solutions at the indicated concentrations in a spot-on-lawn assay. 10 μl of each nisin standard was spotted onto agar plates and, after drying, overlaid with a soft agar containing the indicator strain C. glutamicum CR099. (B) Spot-on-lawn assay with TCA-precipitated supernatant proteins of C. glutamicum CR099/pEKEx2 or C. glutamicum CR099/pEKEx-nisZBTC^{opt}. 10 μl of the fractions were either spotted prior to or after treatment with trypsin. As a positive control, 10 μl of a 125 μg/ml nisin standard was spotted.

FIG. 11 shows (A) Plasmid map of pNZ-Pₙᵢₛ-mCherry^{Lla} introduced into L. lactis NZ9000. (B) A photo of the bacterial pellet taken after growth in GM17 medium containing 10 ng/ml nisin. The pellet already showed reddish color indicating efficient mCherry expression. (C) The recombinant strain L. lactis NZ9000/pNZ-Pₙᵢₛ-mCherry^{Lla} displays red fluorescence (fluorescence units per OD; FLU/OD) in the presence of nisin in a dose-dependent manner and with a limit of detection of less than 0.2-0.5 ng/ml of nisin.

FIG. 12 shows (A) Reverse phase chromatography of the peak fractions obtained by cation exchange chromatography of ammonium sulphate precipitated supernatant proteins of C. glutamicum CR099/pPBEx-nisZBTC^{opt} (left panel) or CR099/pXMJ-nisZBTC^{opt} (right panel) grown in 2xTY medium with glucose (2% w/v) and IPTG (0.1 mM). Black lines indicate absorbance at 214 nm in milli arbitrary units (mAU) and grey lines % of solution B in the elation buffer. Broken lines indicate the boundaries of the peak fractions collected for further analysis. (B) L. lactis NZ9000/pNZ-Pₙᵢₛ-mCherry^{Lla} was used to assay presence of nisin in different samples during purification of ammonium sulphate-precipitated supernatants of C. glutamicum CR099/pXMJ-nisZBTC^{opt} (SN: plain culture supernatant; precip: ammonium sulphate precipitated supernatant proteins; IEX: peak fraction of ion exchange chromatography; RP peak fraction of reverse phase chromatography. pos: 10 ng/ml nisin; +/−: activation by trypsin).

FIG. 13 shows a graphical representation of the genetic constructs for the different approaches that are planned to achieve production of class IIB and IID bacteriocins using recombinant C. glutamicum strains. As an example, constructs for production of garvicin Q (A-D) or flavucin (E) are shown. (A) The gene for garvicin Q (garQ) will be cloned upstream of pedCD encoding the pediocin export apparatus. (B) A synthetic garQ gene encoding the pediocin leader peptide (also referred to here as the signal peptide, SP) fused to the garvicin Q coding sequence and replacing the garvicin Q leader peptide will be cloned upstream of pedCD. (C) The entire gar operon for garvicin Q and its native export apparatus will be cloned. (D) A synthetic gene for a hybrid pre-peptide consisting of any Sec-dependent SP will be cloned upstream of mature garvicin Q. Such pre-peptides will be secreted by the general Sec-dependent protein secretion system. (E) A synthetic gene for a hybrid pre-flavucin consisting of the SP of nisin fused to the sequence for the lantibiotic flavucin of Corynebacterium lipophiloflavum will be cloned upstream of nisBTC encoding the modification and transport proteins of nisin. All constructs will be cloned into pEKEx2, pXMJ-19 or pPBEx2 (not shown), or any other standard C. glutamicum expression vector, downstream of the IPTG-inducible P_{tac} promoter of these plasmids.

FIG. 14 shows establishment of recombinant garvicin Q production by C. glutamicum. (A) Plasmid map of pPBEx2-garQICD^{Cgf} introduced into C. glutamicum CR099 for recombinant production of garvicin Q. (B) Antimicrobial activity in C. glutamicum CR099/pPBEx2-garQICD_{Cgf} and the empty vector control strain CR099/pPBEx2. Activity was analyzed using the fluorescent biosensor Listeria innocua LMG2785/pNZ-Pₕₑₗₚ-pHluorin^{Lm} assay and is indicated by a drop in the ratio of fluorescence intensities (emission at 510 nm) of the strain in LMB buffer after excitation at 400 and 470 nm (ratio RFU 400/470) following treatment with supernatants. Values are mean+/s standard deviation of supernatants obtained from n=3 independent cultures for each strain.

FIG. 15 shows establishment of Sec-dependent production of garvicin Q by C. glutamicum. (A) Plasmid map of pXMJ-SP_{ywaD}-garQ^{Cgf} introduced into C. glutamicum CR099 for recombinant production of garvicin Q via the Sec translocon. (B) Antimicrobial activity in supernatants of C. glutamicum CR099/pXMJ-SP_{ywaD}-garQ^{Cgf} (dark grey bars) and CR099/pPBEx2-garQICD^{Cgf} (lighter grey bars). Activity was analyzed using the fluorescent biosensor Listeria innocua LMG2785/pNZ-Pₕₑₗₚ-pHluorin^{Lm} assay and is indicated by a drop in the ratio of fluorescence intensities (emission at 520 nm) of the strain in LMB buffer after excitation at 400 and 480 nm (ratio Em 520 Ex400/Ex480) following treatment with supernatants. Values are mean +/s standard deviation of supernatants obtained from n=3 independent cultures for each strain.

FIG. 16 shows establishment of flavucin production by C. glutamicum using the nisin biosynthetic machinery. (A) Plasmid map of pXMJ-SPₙᵢₛflaA-nizBTC^{Cgf} introduced into C. glutamicum CR099 for recombinant production of pre-flavucin. (B) Growth inhibition of C. glutamicum CR099/pXMJ19 by supernatants of strains CR099/pXMJ-SPₙᵢₛflaA-nizBTC^{Cgf} and CR099/pXMJ-nisZBTC^{Cgf}. 10 μl of each supernatant were spotted onto agar plates and, after drying, overlaid with a soft agar containing the indicator strain. Where indicated supernatants were incubated with either trypsin or a soluble NisP protease. As a positive control 10 μl of a nisin standard (250 μg/ml) was spotted.

### EXAMPLES

### Example 1—Production of Pediocin in C. glutamicum

C. glutamicum is a Suitable Host for Recombinant Production of Pediocin PA-1

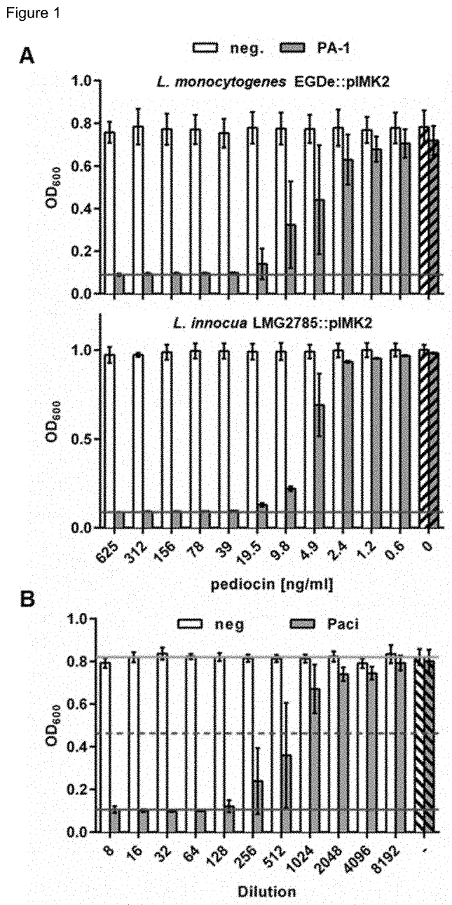

Pediocin PA-1 is a bacteriocin with potent antimicrobial activity against a range of Gram-positive microorganisms including important human pathogens such as Listeria monocytogenes. As a first step towards recombinant production of pediocin PA-1, a growth-dependent assay was established using L. monocytogenes EGDe::pIMK2 or L. innocua LMG2785::pIMK2 as indicator strains, and commercially available, purified pediocin (FIG. 1A). Using serial, 2-fold dilutions of a pediocin standard, 39 ng/ml of pediocin was determined as the minimal concentration to completely inhibit growth of the indicator strains under the conditions of the assay. This concentration was used in further experiments as a calibrator to estimate product concentrations in supernatants of producer strains.

To validate the assay, anti-microbial activity in supernatants of P. acidilactici 347, a natural producer of pediocin was measured following growth in MRS medium to early stationary growth phase (FIG. 1B). Complete inhibition of the indicator strain was achieved with a 1:128 dilution. Thus, supernatants of P. acidilactici 347 contain at least 5 μg mL⁻¹ of active pediocin based on the calibration with the pediocin standard (FIG. 1A). According to a previously published method, activity is calculated as bacteriocin units per ml (BU mL⁻¹) based on the highest dilution showing 50% inhibition of growth of the indicator. According to this definition, supernatants of P. acidilactici 347 contain 10240 BU mL⁻¹.

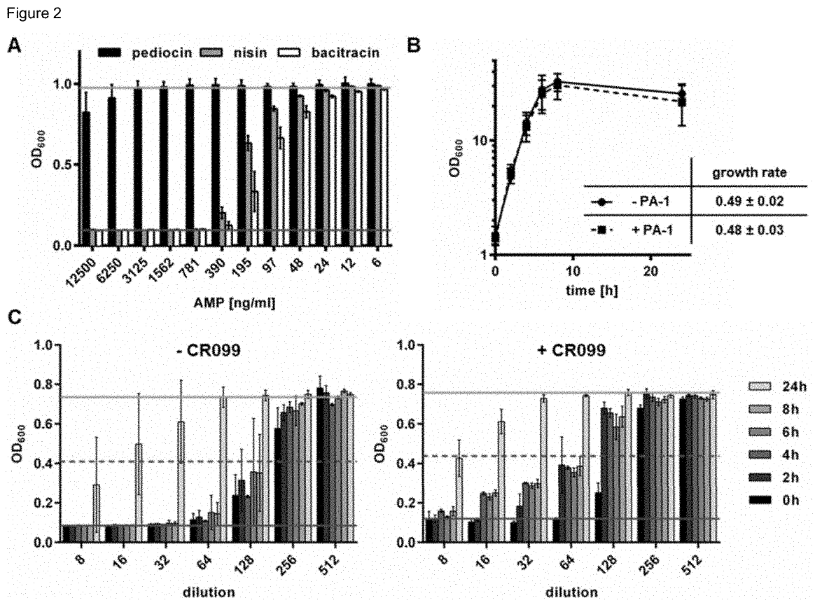

The receptors for bacteriocins of the pediocin family on target organisms are the IIC and IID subunits of a sub-family of mannose-specific phosphotransferase systems (PTS^{Man}; PMID: 19477899). In silico analysis of the genome of C. glutamicum CR099 and other strains of the species indicated that C. glutamicum does not encode a PTS^{Man}, suggesting it may be resistant against pediocin. To corroborate this assumption, the resistance of C. glutamicum CR099 against different antimicrobial peptides was determined using an endpoint measurement of growth in 96-well microtiter plates (FIG. 2A).

Complete inhibition of growth was observed with bacitracin and nisin at concentrations of 390-781 ng mL⁻¹. By contrast, C. glutamicum CR099 was able to grow in the presence of at least 12.5 μg mL⁻¹ pediocin PA-1. Further experiments conducted in larger volumes under standard conditions of cultivation in baffled Erlenmeyer flask confirmed that 2 μg mL⁻¹ of pediocin did not affect final optical density and growth rate (FIG. 2B). This indicates that production of pediocin by C. glutamicum CR099 at significant titers may be possible without adverse effects on growth.

Additionally, activity of pediocin was measured in the culture supernatant at select timepoints during the cultivation. Calculation of bacteriocin units revealed that, after a slight initial reduction from 2560 to 1280 BU mL⁻¹, activity remained more or less stable for several hours in growing C. glutamicum CR099 cultures. After 24 hours, activities dropped to 160 BU mL⁻¹ in the presence of C. glutamicum CR099 and the same reduction was observed in control incubation in 2xTY without bacteria. C. glutamicum does not show significant extracellular protease activity and pediocin activity is known to be sensitive to oxygen. The observed reduction in activity was therefore considered to be related to adsorption of the positively charged pediocin to the negative surface of bacteria and, at later timepoints, to oxidative inactivation. In summary, C. glutamicum CR099 was able to grow in the presence of high concentrations of pediocin and apparently did not degrade pediocin in significant amounts during active growth and was thus considered a suitable host for recombinant production.

Establishment of Pediocin Production in C. glutamicum CR099

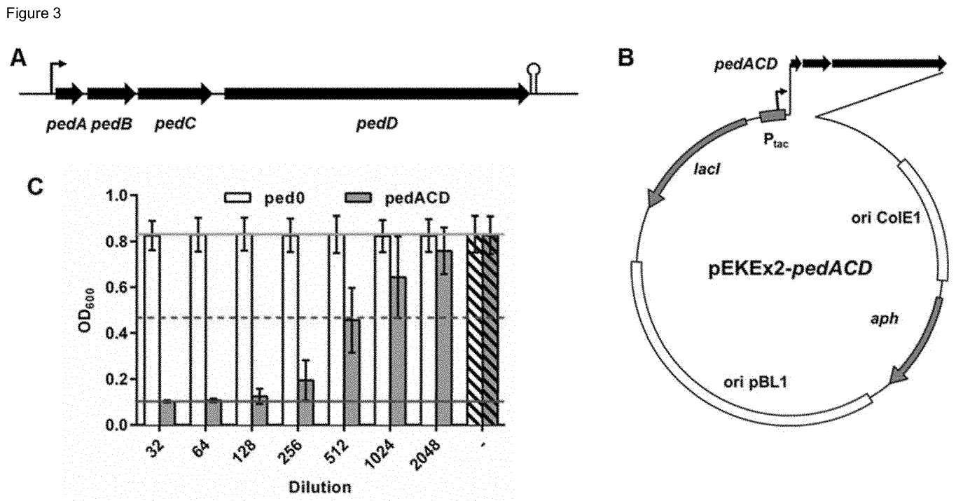

To establish pediocin production in C. glutamicum CR099, the sequence of the biosynthetic operon of P. acidilactici PAC1.0 was retrieved from the NCBI GenBank database (accession no.: M83924.1; FIG. 3A). The operon comprises four genes located on a plasmid (pSRQ11) and consists of the structural gene pedA for the prepeptide of the bacteriocin, pedB for an immunity protein, and pedC and pedD encoding proteins required for processing, cleavage and export of the mature bacteriocin.

In P. acidilactici PAC1.0, all genes are transcribed from a single promoter upstream of pedA. The immunity protein confers resistance by a mechanism that depends on the receptor (PTS^{Man}), which is absent in C. glutamicum CR099. Thus, pedB was considered dispensable. A synthetic pedACD operon for recombinant production of pediocin PA-1 was designed based on the protein sequences available on UniProt database (accession no.: P29430, P37249, and P36497). Gene sequences were codon-optimized for C. glutamicum, each equipped with a ribosome binding site, obtained as synthesized gene fragments, and cloned under the IPTG-inducible Ptac promoter into pEKEx2 by Gibson Assembly yielding pEKEx2-pedACD (FIG. 3B). The codon-optimised gene sequences for pedA, pedC and pedD respectively are shown in SEQ ID NOs 1, 5, and 7. This construct was successfully introduced into C. glutamicum CR099.

Supernatants of cultures of C. glutamicum CR099/pEKEx-pedACD grown to early stationary growth phase in 2xTY medium with 2% Glc and 0.1 mM IPTG in glass tubes contained up to 10240 BU mL⁻¹ of antimicrobial activity against L. monocytogenes corresponding to a concentration of approx. 5 μg mL⁻¹ of pure pediocin (FIG. 3C), i.e. levels comparable to those of the natural producer P. acidilactici 347 (FIG. 1B). By contrast, supernatants of the empty vector control strain C. glutamicum CR099/pEKEx-ped0 did not inhibit growth of L. monocytogenes EGDe::pIMK2 at all. Almost identical results were obtained with a second strain which contained the same operon and promoter in the pXMJ19 backbone.

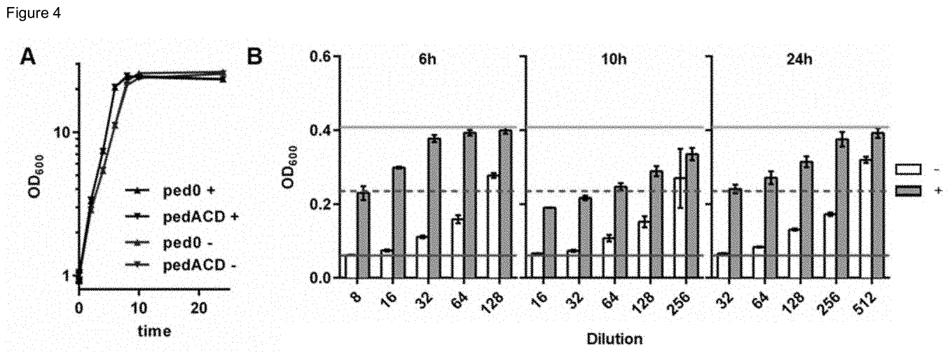

In order to identify the compound responsible for inhibition of growth of the sensor strain and demonstrate that it is indeed pediocin, further experiments in larger culture volumes were conducted. Surprisingly, only very low activity was obtained when C. glutamicum CR099/pEKEx-pedACD was grown in baffled Erlenmeyer flasks for efficient oxygenation of the medium with a maximum of 640 BU mL⁻¹ after 10 h of cultivation (FIG. 4).

However, growth inhibitory activity of C. glutamicum CR099/pEKEx-pedACD supernatants was dramatically increased when cultivated under the same conditions in non-baffled Erlenmeyer flasks. Under these conditions, a maximum of 5120 BU mL-1 (equivalent to approx. 2.5 μg mL⁻¹ of pure pediocin) was observed at the end of the cultivation; FIG. 4B).

Identification of Pediocin in Supernatants of Recombinant C. glutamicum

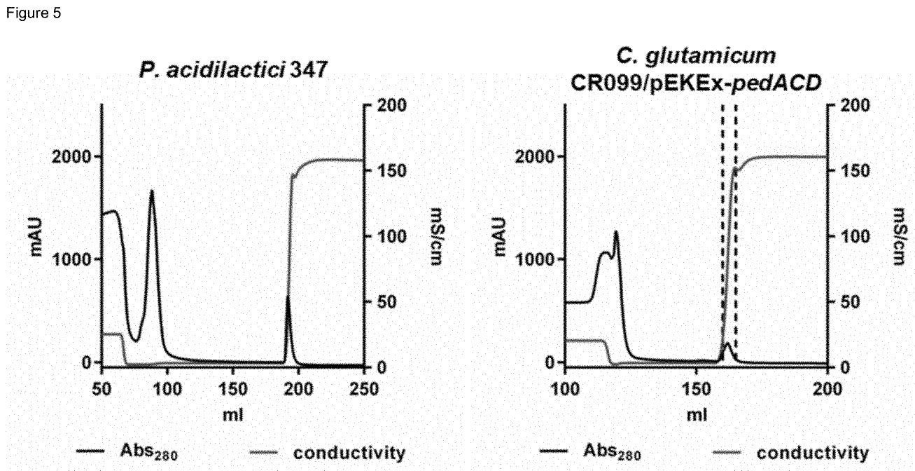

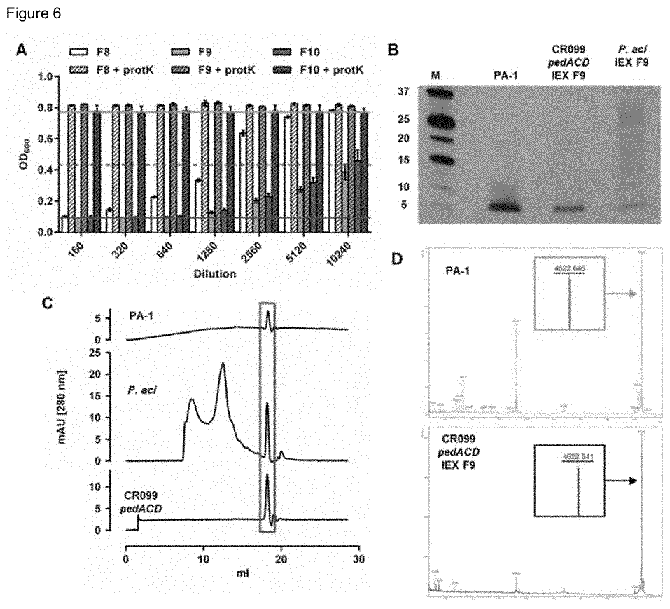

For identification of pediocin, proteins in the supernatant of C. glutamicum CR099/pEKEx-pedACD grown over night in non-baffled Erlenmeyer flasks in 2xTY medium with 2% Glc and 0.1 mM IPTG were precipitated with 50% ammonium sulfate and further separated via cation exchange chromatography (CIEX). Similar to supernatants of P. acidilactici 347, a single peak was observed at 280 nm the onset of elution (FIG. 5). Peak fractions F8-F10 were collected and further analyzed. All peak fractions strongly inhibited growth of L. monocytogenes EGDe::pIMK2 with a maximum of 204,800 BU mL⁻¹, which is equivalent to at least 25 μg mL⁻¹ of active pediocin (FIG. 6A). In all fractions, activity was completely abolished by proteinase K treatment indicating that the active compound is a proteinaceous substance.

Additionally, SDS-PAGE revealed a single protein band in peak fractions of both P. acidilactici 347 and C. glutamicum CR099/pEKEx2-pedACD at around 5 kDa, which corresponds to the size of pediocin PA-1 (FIG. 6B). Size exclusion analysis of cation exchange chromatographic preparations of P. acidilactici 347 and C. glutamicum CR099/pEKEx2-pedACD supernatants revealed a single peak, which is identical to the elution volume of pure, commercially available pediocin (FIG. 6C).

Interestingly, both SDS-PAGE and size exclusion chromatography suggested that the preparations of C. glutamicum CR099/pEKEx2-pedACD contained pediocin as main product in high purity as indicated by a single band or peak. By contrast, samples prepared from P. acidilactici 347 supernatants contained several other peaks or signals indicative of further proteins/peptides possibly secreted by P. acidilactici 347 or present in MRS medium used for cultivation. In order to confirm the active compound, CIEX peak fractions were further analysed by reverse phase chromatography coupled to mass spectrometry (FIG. 6D). This identified a peptide in the supernatants of C. glutamicum CR099/pEKEx2-pedACD with an almost identical mass-to-charge ratio (m/z=4622.891) as pure pediocin PA-1 (m/z=4622.452).

Gene Complement and Order for Efficient Production of Pediocin by C. glutamicum

In order to assess the minimal operon for pediocin production by C. glutamicum, further synthetic constructs with ped genes in different combinations were cloned into pEKEx2 and corresponding plasmids were transformed into C. glutamicum CR099.

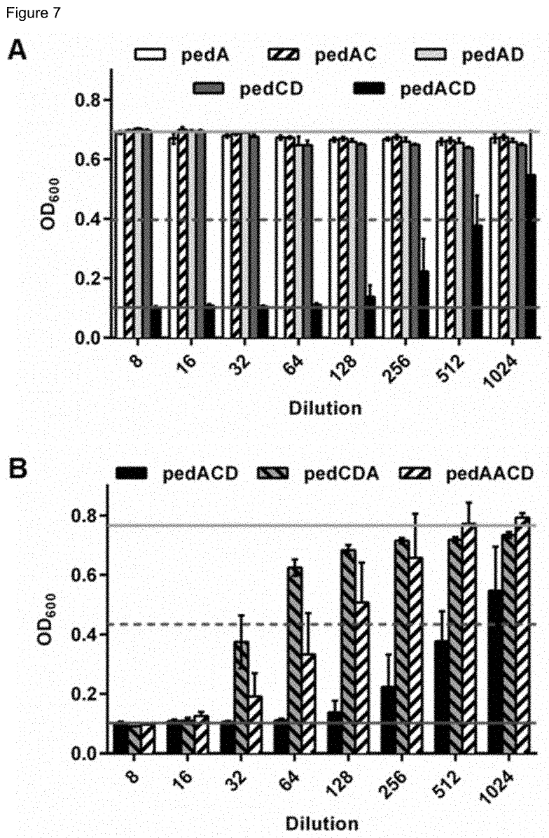

Measurements of activity in supernatants of strains with different combinations of the ped genes (pedA, pedAC, pedAD, pedCD, pedACD) using L. monocytogenes EGDe as sensor strain revealed that strains that lack any of the three genes did not contain pediocin activity in their supernatants (FIG. 7A).

Similarly, altering the gene order by moving the structural gene pedA to the end of the operon (pedCDA) or adding an additional copy of pedA (pedAACD) resulted in reduced activity in the supernatants of the respective strains (FIG. 7B).

### Production of Pediocin in Bioreactors

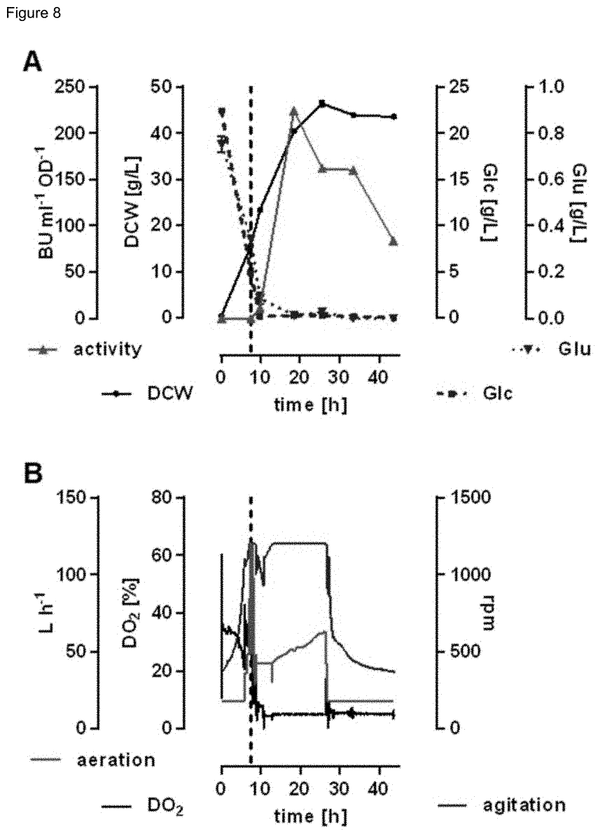

In order to demonstrate feasibility of recombinant pediocin production on a larger scale, further experiments were performed with C. glutamicum CR099/pEKEx-pedACD in bioreactors under fed-batch conditions (FIG. 8). Media composition and culture conditions were similar to the shake flask experiments described above. During the growth phase (0-7.5 h) provided nutrients are consumed and biomass grew at an estimated growth rate of 0.5 h⁻¹ (FIG. 8A), which was in good correspondence to the results of experiments in shake flasks (FIGS. 2 and 4). After 7.5 h, initial Glc was depleted and constant feed addition was started (50 mL h⁻¹ and 1 L of feed) and production was induced (0.1 mM IPTG). During the induction phase, dissolved oxygen was reduced to 5% to prevent exhaustive oxidation of pediocin as indicated by baffled shake flask experiments (FIG. 4). Dissolved oxygen was tightly controlled by a split range control including stirrer speed (400-1200 rpm) and subsequent aeration rate adaption (18-80 L h⁻¹; FIG. 8B). During induction, the fed glucose and glutamate were fully consumed and biomass was formed with a decreased growth rate (0.1 h⁻¹ on average) before growth stopped at a biomass concentration of 46 g L⁻¹ and after complete addition of nutrients (FIG. 8A). No significant amounts of other byproducts such as lactate or acetate were detected. Measurements of pediocin activity indicated active pediocin already 2.5 h after induction reaching a constant and high level of 20480 BU mL⁻¹ at t=18.5-33.5 h of the experiment. This activity corresponds to approx. 10 μg mL⁻¹ of pure pediocin. A slight decrease in activity (10240 BU mL⁻¹) was observed at the end of the experiment (t=43.5 h).

Specific activity reached a maximum of 224.4 BU mL⁻¹ OD⁻¹ at t=18.5 h, i.e. 11 h after induction. This was in good agreement with the specific activities observed at the end (t=24 h) of the experiments in non-baffled Erlenmeyer flasks (200.9 BU mL⁻¹ OD⁻¹).

### Example 2—Production of (Pre)Nisin in C. glutamicum

Preliminary experiments showed that growth of C. glutamicum CR099 is inhibited by nisin at concentrations above ˜100 ng mL⁻¹ (FIG. 2). However, nisin is produced as prenisin, i.e. an inactive precursor of nisin. In the native producer, prenisin is activated after export by a dedicated protease NisP. However, prenisin can also be activated by treatment with the protease trypsin. A strategy for production of prenisin and subsequent activation by trypsin was adopted. To establish prenisin production in C. glutamicum CR099, the sequence of the nisin biosynthesis operon of L. lactis B1629 was obtained by genome sequencing. A synthetic operon encompassing the gene for the nisin Z precursor peptide (nisZ), a dehydratase for modification (nisB), a cyclase for formation of the thiolactone rings (nisC), and a dedicated transporter for the modified prepeptide (nisT) was designed based on the deduced protein sequences. Gene sequences were codon-optimized for C. glutamicum, each equipped with a ribosome binding site, obtained as synthesized gene fragments, and cloned under the IPTG-inducible P_{tac} promoter into pEKEx2, pPBEx2 or pXMJ19 by Gibson Assembly yielding pEKEx-nisZBTC^{opt} pPBEx-nisZBTC^{opt} and pXMJ-nisZBTC^{opt}. These constructs were successfully introduced into C. glutamicum CR099.

Supernatants of cultures of the recombinant strains C. glutamicum CR099/pEKEx-nisZBTC^{opt} CR099/pPBEx-nisZBTC^{opt} and CR099/pXMJ-nisZBTC^{opt} were grown to early stationary growth phase in 2xTY medium with 2% glucose and 0.1 mM IPTG. Proteins were precipitated using ammonium sulphate and the precipitates were washed using ice cold acetone, resuspended in Tris/HCl buffer (pH 6.5) and analysed by ion exchange chromatography (FIG. 1). A single peak was observed in all samples at 214 nm the onset of elution.

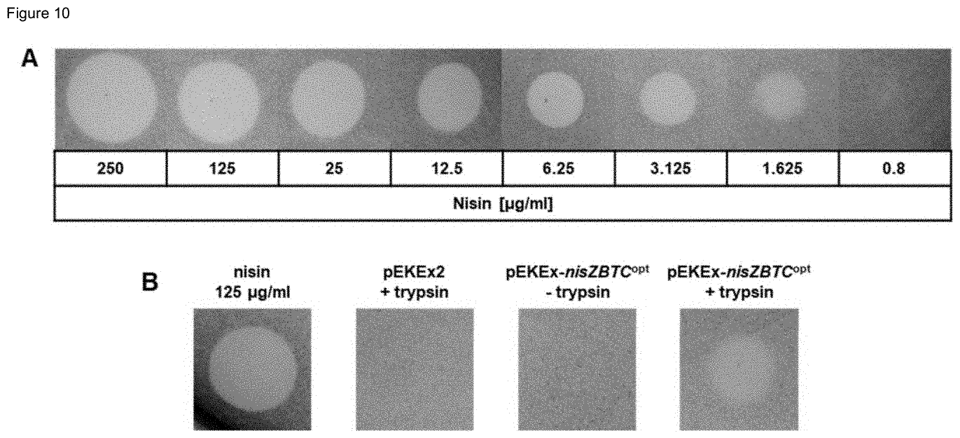

Using a spot-on-lawn using C. glutamicum CR099 as an indicator strain (FIG. 10), no inhibition of growth was observed for proteins precipitated from the negative control, i.e. supernatants of the empty vector control strain C. glutamicum CR099/pEKEx2. However, the proteins precipitated from supernatants of C. glutamicum CR099/pEKEx-nisZBTC^{opt} showed inhibitory activity of the indicator strain after treatment with trypsin (FIG. 10B) to a similar extent as a nisin standard at a concentration of 1-2 μg mL⁻¹ (FIG. 10A). By contrast, the same protein preparation without further treatment did not inhibit growth of the indicator strain. This indicates that trypsin treatment was able to activate a compound present in the supernatants of C. glutamicum CR099/pEKEx-nisZBTC^{opt}.

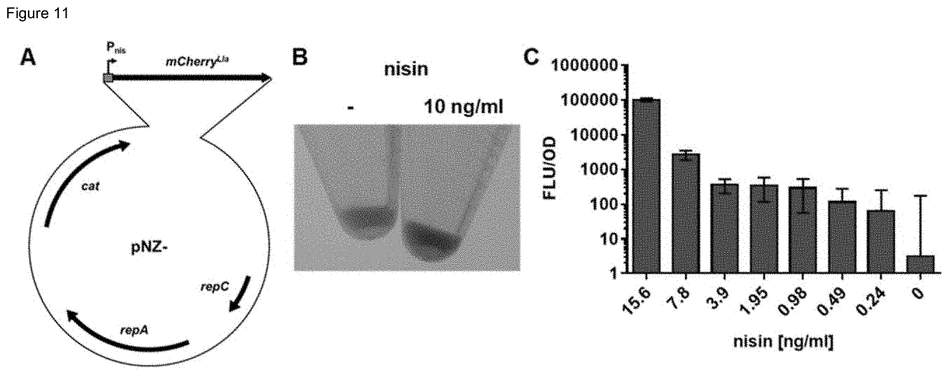

The sensitivity of the spot-on-lawn assay using C. glutamicum CR099 as an indicator strain is very low. In order to establish a sensor system with higher sensitivity and to demonstrate production of prenisin by C. glutamicum CR099/pEKEx-nisZBTC^{opt}, a L. lactis sensor strain was generated. The strain L. lactis NZ9000/pNZ-Pₙᵢₛ-mCherry^{Lla} contains the NisRK two-component system and harbours a pNZ-derives plasmid for expression the red-fluorescent protein mCherry from the Pₙᵢₛ promoter (FIG. 11A), which is activated by NisR in a strictly nisin-dependent manner. In this construct, the coding sequence of the mCherry protein is optimized for codon usage of L. lactis. The respective sensor strain results in reddish coloured bacteria after o/N growth in the presence of 10 ng mL⁻¹ (FIG. 11B). In a 96-well microtiter plate assay, the limit of detection of this sensor strain was 0.25-0.5 ng mL⁻¹ of active nisin (FIG. 11C).

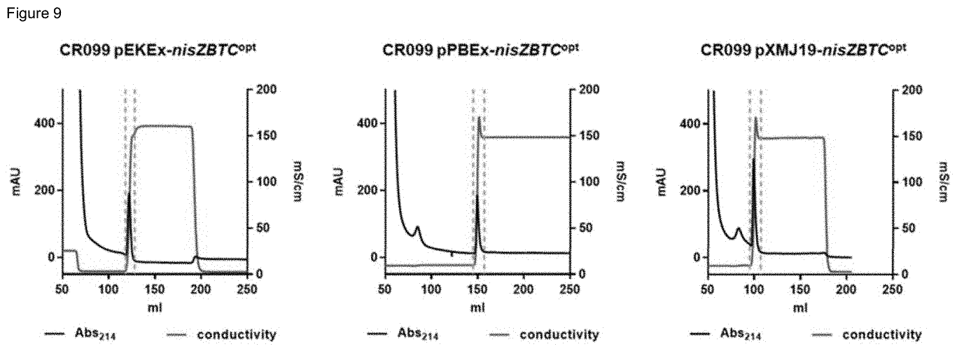

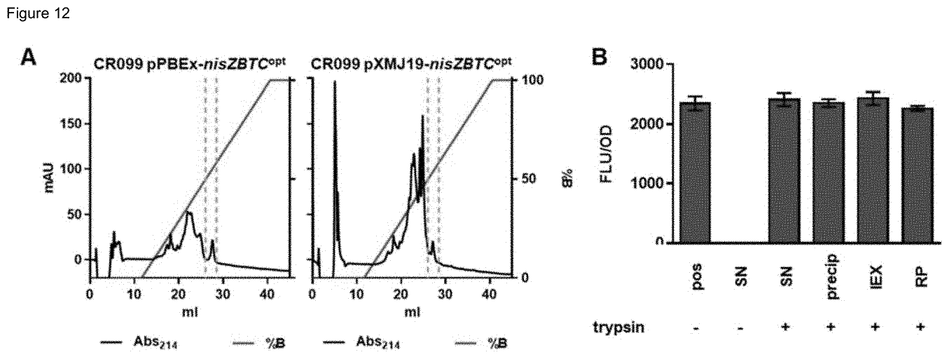

The peak fraction of cation exchange chromatography of supernatant proteins precipitated by ammonium sulphate (FIG. 9) were further analysed by reverse phase chromatography. This revealed distinct peaks (FIG. 12A) that were absent in samples of the empty vector control strains. These peak fractions were collected and were clearly able to activate expression of mCherry in L. lactis NZ9000/pNZ-Pₙᵢₛ-mCherry^{Lla} following trypsin treatment (FIG. 12B). Similarly, peak fractions of cation exchange chromatography, precipitated supernatant proteins and plain supernatants treated with trypsin were able to induce mCherry expression in the sensor strain. By contrast, the sensor strain did not show any mCherry fluorescence when exposed to untreated plain culture supernatants.

Considering the signal intensity of the mCherry sensor, the dilution of samples in the assay and the signal intensity of the sensor treated with 10 ng mL⁻¹ of nisin, all trypsin-activated samples contained at least 2 μg mL⁻¹ of active nisin.

### Example 3—Production of Further Bacteriocins in C. glutamicum

It has been demonstrated that it is possible to produce the class IIA bacteriocin pediocin PA-1 and the prepeptide of class I bacteriocin nisin using recombinant derivatives of C. glutamicum. Several approaches are possible to implement production of other bacteriocins depending on the class and nature of the peptide. For several class II bacteriocins, especially those that contain a specific double glycine (GG) motif in their leader sequence, it has been shown that the transport and modification machinery is promiscuous. For example, the GG-leader of lactococcin A (class IID) and its dedicated ABC transporter can be used to produce pediocin (class IIA). Fusion of the pediocin leader sequence to the coding sequence of colicin V allowed production of active colicin in a strain harbouring this construct and the genes required for pediocin secretion. Similarly, active divergicin A (class IIA) could be produced by fusion of the GG-leaders of leucocin A (class IIA), lactococcin A (class IID) or colicin V (unclassified). Thus, it is expected that class II bacteriocins that carry a GG motif in their leader sequence can be produced using the pediocin export apparatus (or the export apparatus of other bacteriocins with a GG-leader). Potential candidates amongst class II bacteriocins include pediocin, lactococcin G (class IIB), plantaricin EF (class IIB), plantaricin JK (class IIB), plantaricin NC08 (class IIB), lactococcin A (class IID), lactococcin B (class IID), and garvicin Q (class IID).

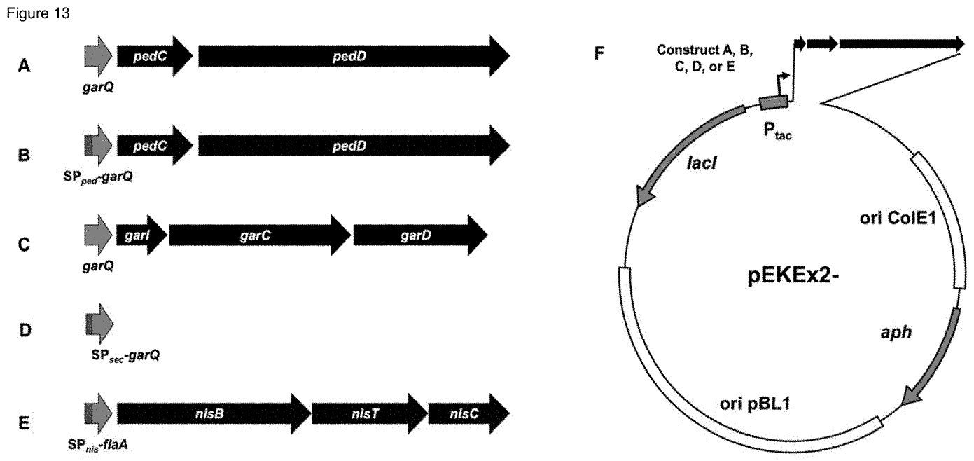

For all these bacteriocins, natural producers will be cultivated in standard media and supernatants will be tested for antimicrobial activity. These experiments will be carried out with bacteria that are shown to be sensitive to the bacteriocin to ensure that an active bacteriocin is present in the supernatants. Additionally, antimicrobial activity against C. glutamicum CR099 will be tested to assess toxicity of the product towards the anticipated production host. In a first round, only bacteriocins that do not inhibit growth of C. glutamicum CR099 will be taken into consideration for generation of recombinant producers. The coding sequences of these bacteriocins will be obtained as synthetic DNA constructs with sequences optimized for codon usage of C. glutamicum. In a first approach, these sequences will be cloned into the plasmids generated for pediocin production (pEKEx-pedACD, pXMJ-pedACD) replacing the pedA gene (FIG. 13A, shown for garvicin Q as the bacteriocin of interest by way of example). This illustrates expression of a bacteriocin which is processed and exported using the processing and transport genes of another bacteriocin. Alternative approaches for production of bacteriocins (not limited to class II bacteriocins) include:

a) the generation of chimeric bacteriocins consisting of the coding sequences of the leader sequence of one bacteriocin fused to another bacteriocin (FIG. 13B, shown for garvicin Q by way of example using a class II bacteriocin leader, e.g. the pediocin leader sequence/signal peptide);

b) cloning of the bacteriocin gene along with its native processing and export apparatus in a similar fashion as carried out for pediocin production strains (FIG. 13C, shown for garvicin Q by way of example);

c) expression of a hybrid pre-peptide consisting of any Sec-dependent signal peptide (SP) and mature bacteriocin. Such pre-peptides will be secreted by the general Sec-dependent protein secretion system and so not require specific transporters (FIG. 13D, shown for garvicin Q by way of example);

d) generation of a gene for a hybrid/chimeric bacteriocin consisting of the SP/leader sequence of a second bacteriocin (e.g. nisin) fused to the sequence for the bacteriocin of interest which is to be expressed. The corresponding synthetic gene of the bacteriocin of interest will be cloned upstream of the genes encoding the modification and transport proteins of the second bacteriocin (e.g. nisin, i.e. using NisBTC) allowing modification and secretion of the bacteriocin of interest with the biosynthetic machinery of the second bacteriocin. (FIG. 13E, shown for the lantibiotic flavucin of Corynebacterium lipophiloflavum, by way of example).

Lactococcin A and B and garvicin Q are single peptide bacteriocins. Thus, generation of the respective recombinant C. glutamicum strains is expected to be relatively straight forward with a plasmid containing the structural gene and genes for the export apparatus in one of the described setups resulting in a single producer. The class IIB bacteriocins lactococcin G and plantaricins EF, JK, and NC08 are two-peptide bacteriocins that require interaction of both peptides in specific molar ratios. For example, lactococcin G is fully active in complexes of α and β peptides in a molar ratio of 7:1 or 8:1 respectively 4. Here, separate producer strains will be generated for each of the peptides and bacteriocins will be produced separately, purified by downstream processing. The two peptides will then be combined to an optimized formula containing both peptides on molar ratios that ensures maximum activity.

### Example 4—Production of Garvicin Q in C. glutamicum Using the GarCD Transporter

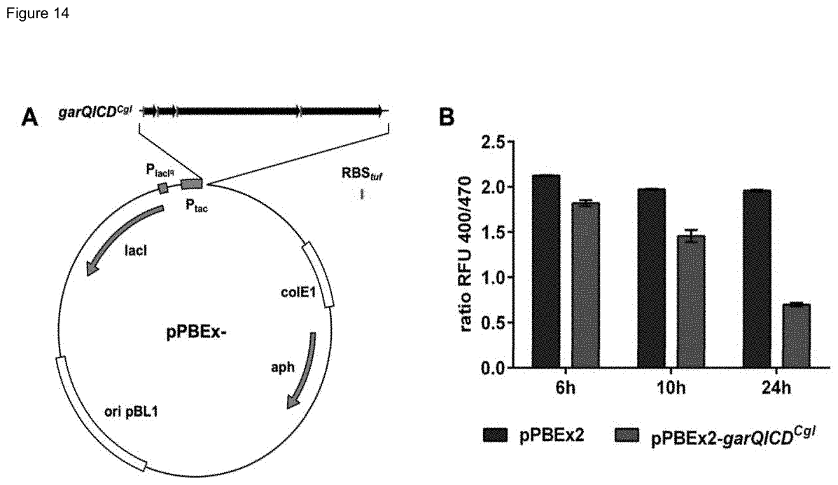

This Example illustrates the general methodology of cloning the bacteriocin gene along with its native export apparatus, using garvicin Q as the bacteriocin. The genetic construct used for this purpose is shown in FIGS. 13C and 14A.

GarQ is a class IID bacteriocin consisting of a single linear peptide that is produced by different strains of Lactococcus garvieae. The receptors of pediocin and GarQ are identical and were show to be IIC and IID subunits of group I mannose-family phosphotransferase system (PTS^{Man}). C. glutamicum CR099 lacks a PTS^{Man} and is therefore expected to be resistant to GarQ.

To establish GarQ production in C. glutamicum CR099, the sequence of the biosynthetic operon for GarQ was retrieved from the natural producer Lactococcus garvieae B1726. The operon comprises four genes consisting of the structural gene garQ for the prepeptide of the bacteriocin, garI for an immunity protein, and garC and garD encoding proteins that are probably required for processing, cleavage and export of the mature bacteriocin. A synthetic garQICD^{Cgl} operon (^{Cgl}: codon-optimized for C. glutamicum) for recombinant production of GarQ was designed with gene sequences codon-optimized for C. glutamicum, each equipped with a ribosome binding site, obtained as synthesized gene fragments, and cloned under the IPTG-inducible P_{tac} promoter into pPBEx2 (Bakkes et al., 2020, supra) by Gibson Assembly yielding pPBEx-garQICD^{Cgl}(FIG. 14). This construct was successfully introduced into C. glutamicum CR099. The codon optimized sequences for the genes Gar Q, I, C and D are shown in SEQ ID NOs. 75-78.

TABLE 1
Bacterial strains and plasmids used in this example.

| | Relevant characteristics | Source |
|---|---|---|
| Strain | | |

Corynebacterium glutamicum

| | | |
|---|---|---|
| CR099 | C. glutamicum ATCC 13032, | Baumgart et al. |
| | ΔCGP1, ΔCGP2, ΔCGP3, | Appl. Environ. |
| | ΔISCg1, ΔISCg2 | Microb. |
| | | (2013):79(19):6006-15 |

Listeria innocua

| | | |
|---|---|---|
| LMG2785 | indicator strain | unpublished |
| Plasmid | | |
| pNZ-Pₕₑₗₚ- | Reporter plasmid for pHluro | Crauwels et al., |
| pHluorin^{Lm} | assays to determine antimicrobial | Front. Microbiol. |
| | activity in supernatants of | (2018):9:3038 |
| | bacteriocin producers | |
| pPBEx2 | E. coli/C. glutamicum shuttle | Bakkes et al., |
| | vector; Ptacl; lacl^{q}; oriC.g | 2020, supra |
| | from pBL1.; oriE.c. ColE1 from | |
| | pUC18; Kan^{r}. | |
| pPBEx- | pPBEx2 derivative for IPTG- | C. Desiderato, |
| garQICD^{Cgl} | inducible expression of the | unpublished |
| | synthetic garicin operon | results |
| | garQICD^{Cgl} of | |
| | Lactococcus garvieae B1726 | |

Supernatants of this strain cultivated in a modified CGXII minimal medium containing 2% Glc and 0.2 mM IPTG contained antimicrobial activity against Listeria innocua LMG2785/pNZ-Pₕₑₗₚ-pHluorin^{Lm}, a recently described fluorescent biosensor for detection of baceriocins with membrane-damaging activity (Desiderato et al., Int. J. Mol. Sci. (2021): 22(16), 8615). This activity increased during cultivation of C. glutamicum CR099/pPBEx2-garQICD^{Cgf} and was absent in supernatants of the empty vector control strain C. glutamicum CR099/pPBEx2 suggesting successful production of garvicin Q (FIG. 14B).

### Example 5—Production of Garvicin Q in C. glutamicum Using Sec-Dependent Protein Secretion

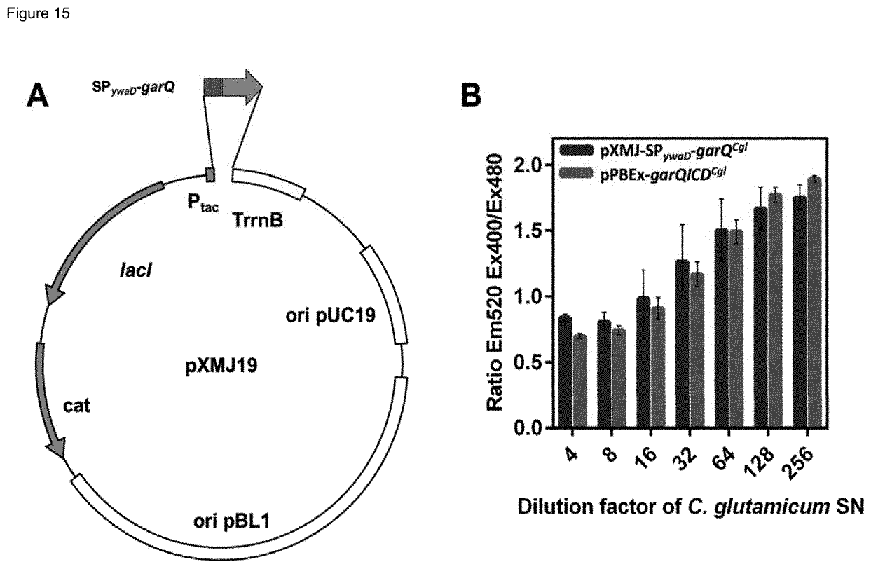

This Example illustrates the general methodology of expression of a hybrid pre-peptide consisting of any Sec-dependent signal peptide (SP) and mature bacteriocin, using garvicin Q as the bacteriocin. The genetic construct used for this purpose is shown in FIGS. 13D and 15A.

In this example a synthetic gene consisting of coding sequences of the Sec-dependent secretion signal of aminopeptidase YwaD of Bacillus subtilis (peptide sequence shown in SEQ ID NO: 79) and mature garvicin Q was generated. This synthetic gene was cloned under the IPTG-inducible P_{tac} promoter into pXMJ19 (Jakoby et al. Biotech. Tech. (1999):13(6):437-41) by Gibson Assembly yielding pXMJ-SP_{ywaD}-garQ (FIG. 15A). This construct was successfully introduced into C. glutamicum CR099.

TABLE 2
Bacterial strains and plasmids used in this example¹.

| Plasmid | Relevant characteristics | Source |
|---|---|---|
| pXMJ19 | E. coli/C. glutamicum shuttle vector; | Jakoby, 1999, |
| | Ptacl; lacl^{q}; oriC.g from pBL1.; | supra |
| | oriE.c. ColE1 from pUC18; Cm^{r}. | |
| pXMJ-SP_{ywaD}- | pXMJ19 derivative for IPTG- | C. Desiderato, |
| garQ^{Cgl} | inducible expression of the | unpublished |
| | synthetic operon garQICD^{Cgl} | results |
| | for production of garvicin Q | |

¹strains CR099 and LMG2785 and plasmids pNZ-Pₕₑₗₚ-pHluorin^{Lm} and pPBEx-garQICD^{Cgl} are as in Table 1.

Supernatants of C. glutamicum CR099/pXMJ-SP_{ywaD}-garQ^{Cgl} cultivated in a modified CGXII minimal medium containing 2% Glc and 0.2 mM IPTG contained antimicrobial activity against Listeria innocua LMG2785/pNZ-Pₕₑₗₚ-pHluorin^{Lm}, as used in Example 4. Activity was comparable to that measured in supernatants of C. glutamicum CR099/pPBEx2-garQICD^{Cgf}, i.e. the strain producing garvicin Q using the garvicin-specific transporters GarCD (FIG. 15B). This demonstrates that active garvicin Q can be produced using a Sec-secretion signal of B. subtilis and the Sec translocon of C. glutamicum.

### Example 6—Production of Flavucin Q in C. glutamicum Using Nisin Modification and Transport Machinery and Downstream Activation

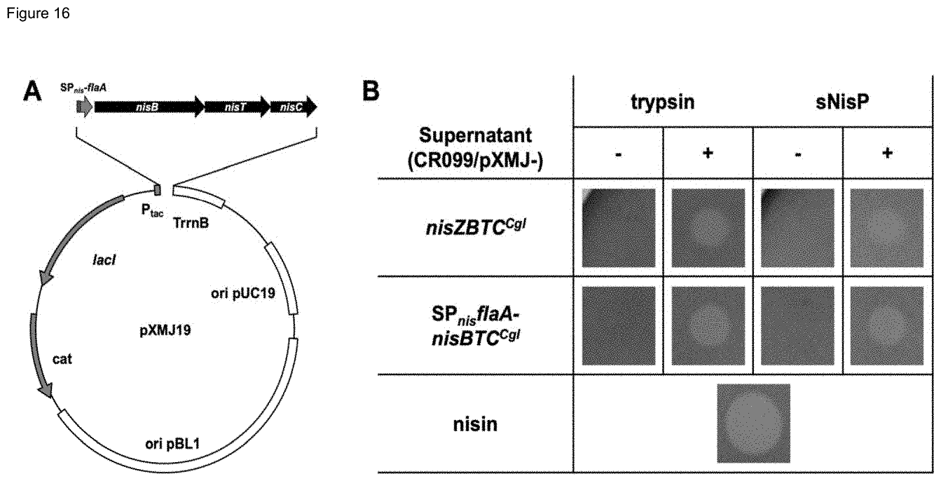

This Example illustrates the general methodology of generation of a gene for a hybrid bacteriocin consisting of the SP of nisin fused to the sequence for the bacteriocin using flavucin as the bacteriocin. The genetic construct used for this purpose is shown in FIGS. 13E and 16A.

For production of a pre-peptide for a class I lanthipeptide using the biosynthetic machinery of nisin, a synthetic gene consisting of coding sequences of the leader peptide of nisin and the core peptide of flavucin, a class I lantibiotic produced by Corybebacterium lipophiloflavum (Van Heel et al. ACS Syn. Biol. (2016):5(1):1146-54), was generated. This synthetic gene was cloned under the IPTG-inducible P_{tac} promoter into pXMJ19 (Jakoby et al., 1999, supra) by Gibson Assembly yielding pXMJ-SPₙᵢₛflaA-nizBTC^{Cgl} (FIG. 16A).

TABLE 3
Bacterial strains and plasmids used in this example.

| Plasmid | Relevant characteristics | Source |
|---|---|---|
| pXMJ-nisZBTC^{Cgl} | pXMJ19 derivative for IPTG- | Weixler et al. |
| | inducible expression of the | Microb. Cell Fact. |
| | synthetic nisin operon | (2022):21(1):11 |
| | nisZBTC^{Cgl} for production | |
| | of pre-nisin | |
| pXMJ-SPₙᵢₛ-flaA- | pXMJ19 derivative for IPTG- | D. Weixler, |
| nisBTC^{Cgl} | inducible expression of the | unpublished |
| | synthetic operon SPₙᵢₛflaA- | results |
| | nisBTC^{Cgl} for production | |
| | of pre-flavucin | |

¹ strain CR099 and plasmid pXMJ19 are as in Tables 1 and 2.

Strains CR099/pXMJ-SPₙᵢₛflaA-nizBTC^{Cgl} and CR099/pXMJ-nisZBTC^{opt} were grown to early stationary growth phase in 2xTY medium with 2% glucose and IPTG (0.1 mM) and supernatants were collected for further analysis. Using a spot-on-lawn using C. glutamicum CR099/pXMJ19 as an indicator strain (FIG. 16B), no inhibition of growth was observed for untreated supernatants. However, inhibitory activity was observed for supernatants of both strains after treatment with trypsin or a soluble version of the NisP protease (sNisP; FIG. 16B). This suggests that both trypsin and sNisP are able to activate the pre-peptides of nisin or flavucin released into the supernatants by C. glutamicum CR099/pXMJ-nisZBTC^{opt} or CR099/pXMJ-SPₙᵢₛflaA-nizBTC^{Cgl}, respectively. These results indicate successful production of fully modified pre-flavucin by C. glutamicum CR099/pXMJ-SPₙᵢₛflaA-nizBTC^{opt} and activation to mature flavucin by sNisP.

### DESCRIPTION OF SEQUENCES—AS PROVIDED IN THE SEQUENCE LISTING

### Pediocin

SEQ ID NO 1: pedA gene codon-optimised
SEQ ID NO 2: PedA protein with leader (from UniProt P29430)
SEQ ID NO 3: PedA protein without leader (from UniProt P29430)
SEQ ID NO 4: PedA leader (from UniProt P29430)
SEQ ID NO 5: pedC gene codon-optimised
SEQ ID NO 6: PedC protein (from UniProt P37249)
SEQ ID NO 7: pedD gene codon-optimised
SEQ ID NO 8: PedD protein (from UniProt 36497)

### Lactococcin G

SEQ ID NO: 9 Alpha with leader
SEQ ID NO: 10 Alpha w/o leader
SEQ ID NO: 11 Beta w leader
SEQ ID NO: 12 Beta w/o leader

### Plantaricin EF

SEQ ID NO: 13 E w leader
SEQ ID NO: 14 E w/o leader
SEQ ID NO: 15 F w leader
SEQ ID NO: 16 F w/o leader

### Plantaricin JK

SEQ ID NO: 17 J w leader
SEQ ID NO: 18 J w/o leader
SEQ ID NO: 19 K w leader
SEQ ID NO: 20 K w/o leader

### Plantaricin NC08

SEQ ID NO: 21 Alpha w leader
SEQ ID NO: 22 Alpha w/o leader
SEQ ID NO: 23 Beta w leader
SEQ ID NO: 24 Beta w/o leader

### Lactococcin A

SEQ ID NO: 25 w leader
SEQ ID NO: 26 w/o leader

### Lactococcin B

SEQ ID NO: 27 w leader
SEQ ID NO: 28 w/o leader

### Garvicin Q

SEQ ID NO: 29 w leader
SEQ ID NO: 30 w/o leader

### Nisin

SEQ ID NO: 31 Nisin Z with leader (from UniProt P29559)
SEQ ID NO: 32 Nisin Z w/o leader (from UniProt P29559)

### Lacticin

SEQ ID NO: 33 IctA with leader (from UniProt P36499)
SEQ ID NO: 34 IctA w/o leader (from UniProt P36499)

### Subtilin

SEQ ID NO: 35 spaS with leader (from UniProt P10946)
SEQ ID NO: 36 spaS w/o leader (from UniProt P10946)

### Epicidin

SEQ ID NO: 37 from UniProt 054220
SEQ ID NO: 38, sequence w/o leader

### Epidermin

SEQ ID NO: 39 epiA with leader (from UniProt P08136)
SEQ ID NO: 40 epiA w/o leader (from UniProt P08136)

### Epilancin

SEQ ID NO: 41 elxA with leader (from UniProt 86047)
SEQ ID NO: 42 elxA w/o leader (from UniProt 86047)

### Sublancin

SEQ ID NO: 43 sunA with leader (from UniProt P68578)
SEQ ID NO: 44 sunA w/o leader (from UniProt P68578)

### Carnocin

SEQ ID NO: 45 cbnB2 with leader (from UniProt P38580)
SEQ ID NO: 46 cbnB2 w/o leader

### Variacin

SEQ ID NO: 47 from UniProt Q50848
SEQ ID NO: 48 sequence w/o leader

### Cypemycin

SEQ ID NO: 49 cypA with leader (from UniProt E5K1B6)
SEQ ID NO: 50 cypA w/o leader(from UniProt E5KIB6)

### Gallidermin

SEQ ID NO: 51 gdmA with leader (from UniProt P21838)
SEQ ID NO: 52 gdmA w/o leader (from UniProt P21838)

### Mersacidin

SEQ ID NO: 53 mrsA with leader (from UniProt P43683)
SEQ ID NO: 54 mrsA w/o leader(from UniProt P43683)

### Actagardine

SEQ ID NO: 55 garA with leader (from UniProt P56650)
SEQ ID NO: 56 garA w/o leader (from UniProt P56650)

### Cinnamycin

SEQ ID NO: 57 cinA w leader (from UniProt P29827)
SEQ ID NO: 58 cinA w/o leader (from UniProt P29827)

### Duramycin

SEQ ID NO: 59 w leader
SEQ ID NO: 60 w/o leader from UniProt P36504

### Ancovenin

SEQ ID NO: 61 w/o leader from UniProt P38655

### Enterococcal Cytolysin

SEQ ID NO: 62 mature ClyLl
SEQ ID NO: 63 Cytolysin_ClyLl with precursor
SEQ ID NO: 64 mature ClyLs
SEQ ID NO: 65 Cytolysin_ClyLs with precursor:

### Staphylococcin C55

SEQ ID NO: 66 Staphylococcins_C55b_SacbA
SEQ ID NO: 67 Staphylococcins_C55a_SacaA

### Mutacin

SEQ ID NO: 68 lanA w leader (from UniProt 68586)
SEQ ID NO: 69 lanA w/o leader (from UniProt 68586)
SEQ ID NO: 70 Consensus sequence for Class HA (wherein Xaa is any aa)
SEQ ID NO: 71 nisZ gene codon optimised
SEQ ID NO: 72 nisB gene codon optimised
SEQ ID NO: 73 nisT gene codon optimised
SEQ ID NO: 74 nisC gene codon optimised
SEQ ID NO: 75 garvicin Q gene codon optimised (garQ^{Cgl})
SEQ ID NO: 76 garvicin I gene codon optimised (garI^{Cgl})
SEQ ID NO: 77 garvicin C gene codon optimised (garC^{Cgl})
SEQ ID NO: 78 garvicin D gene codon optimised (garD^{Cgl})
SEQ ID NO: 79 Sec-dependent secretion signal of aminopeptidase YwaD of Bacillus subtilis (peptide)

1. A method of producing a Class I or Class II bacteriocin, said method comprising:
(a) providing a modified bacterial strain of coryneform bacteria into which has been introduced a heterologous nucleic acid molecule encoding a Class I or Class II bacteriocin polypeptide;
(b) culturing said modified strain under conditions suitable for expression of said bacteriocin polypeptide; and
(c) optionally, harvesting said Class I or Class II bacteriocin polypeptide produced in step (b),
wherein the bacteriocin polypeptide is an inactive precursor, and/or said strain is not susceptible to said bacteriocin and/or said bacteriocin polypeptide is a component polypeptide of a multi-peptide bacteriocin and said modified strain does not produce all other component polypeptides required to make a functional bacteriocin, wherein optionally said bacteriocin is harvested, where preferably the bacteriocin is isolated, purified or processed into a product.

2. (canceled)

3. The method of claim 1, wherein
(a) when said bacteriocin is a Class I bacteriocin, the bacteriocin polypeptide is an inactive precursor, and when said bacteriocin is a class II bacteriocin, said strain is not susceptible to said bacteriocin and/or said bacteriocin polypeptide is a single polypeptide of a multi-peptide bacteriocin;
(b) an expression vector comprising said nucleic acid molecule has been introduced into said modified strain, wherein said expression vector is capable of expressing said bacteriocin polypeptide in said strain; and/or
(c) said nucleic acid molecule comprises a synthetic operon comprising:
(i) a promoter controlling the expression of the following genes;
(ii) a structural gene encoding the bacteriocin polypeptide;
(iii) optionally, one or more genes encoding processing and/or transport proteins for production of said bacteriocin polypeptide; and/or
(iv) optionally, one or more genes which provide the bacterial strain with immunity to the bacteriocin.

4-5. (canceled)

6. The method of claim 35, wherein
(a) said synthetic operon comprises one or more genes encoding processing and/or transport proteins for production of said bacteriocin polypeptide;
(b) said synthetic operon comprises
(i) a promoter controlling the expression of the following genes;
(ii) a structural gene encoding the bacteriocin polypeptide which is a chimeric bacteriocin polypeptide which comprises a heterologous leader sequence which is a leader sequence of a second bacteriocin;
(iii) one or more genes encoding processing and/or transport proteins for production of said bacteriocin polypeptide wherein said genes are processing and/or transport proteins for processing and/or transporting said second bacteriocin; and
(iv) optionally, one or more genes which provide the bacterial strain with immunity to the bacteriocin; or
(c) said synthetic operon comprises
(i) a promoter controlling the expression of the following genes;
(ii) a structural gene encoding the bacteriocin polypeptide which is a chimeric bacteriocin polypeptide which comprises a heterologous leader sequence which is a Sec-dependent leader sequence; and
(iii) optionally, one or more genes which provide the bacterial strain with immunity to the bacteriocin.

7-8. (canceled)

9. The method of claim 3, wherein said nucleic acid molecule comprises a synthetic operon, and wherein said genes are codon-optimised for expression in coryneform bacteria.

10. The method of claim 1, wherein said nucleic acid molecule is a self-replicating plasmid or a plasmid which has been integrated into the genome of the strain.

11. The method of claim 3, wherein said nucleic acid molecule comprises a synthetic operon, and wherein said promoter is an inducible promoter.

12. The method of claim 1, wherein
(a) the strain does not express a protein capable of acting as a receptor for the class I or II bacteriocin to be expressed; and/or
(b) said modified bacterial strain (i) does not contain a gene which provides the bacterial strain with immunity to the bacteriocin or (ii) contains a constitutively expressed gene which provides the bacterial strain with immunity to the bacteriocin.

13. (canceled)

14. The method of claim 1, wherein the Class I or Class II bacteriocin is a Class II bacteriocin.

15. The method of claim 14, wherein
(a) said strain does not express a Group I mannose-specific phosphotransferase (PTS^{Man});
(b) the leader sequence of said Class II bacteriocin comprises a double glycine motif, and/or
(c) the Class II bacteriocin is a Class IIA, Class IIB or Class BD bacteriocin.

16-17. (canceled)

18. The method of claim 14, wherein the bacteriocin is selected from the group consisting of pediocin, lactococcin G, plantaricin EF, plantaricin JK, plantaricin NC08, lactococcin A, lactococcin B and garvicin Q.

19. The method of claim 14, wherein the nucleic acid molecule comprises
(a) a synthetic operon comprising:
(i) a promoter;
(ii) a structural gene encoding the bacteriocin; and
(iii) pedC and pedD genes, wherein preferably the structural gene encodes pediocin, or a chimeric bacteriocin polypeptide which comprises the leader sequence of pediocin;
(b) a synthetic operon comprising:
(i) a promoter;
(ii) a structural gene encoding the bacteriocin;
(iii) garC and garD genes; and
(iv) optionally, one or more genes which provide the bacterial strain with immunity to the bacteriocin, wherein preferably the structural gene encodes garvicin Q, or a chimeric bacteriocin polypeptide which comprises the leader sequence of garvicin Q; or
(c) a synthetic operon comprising:
(i) a promoter;
(ii) a structural gene encoding the bacteriocin polypeptide which is a chimeric bacteriocin polypeptide which comprises a heterologous leader sequence which is a Sec-dependent leader sequence; and
(iii) optionally, one or more genes which provide the bacterial strain with immunity to the bacteriocin, wherein preferably the structural gene encodes garvicin Q.

20-21. (canceled)

22. The method of claim 14, wherein the Class II bacteriocin is a multi-peptide bacteriocin comprising 2 or more bacteriocin polypeptides, and the method comprises separately expressing each bacteriocin polypeptide in the bacterial strain, harvesting each bacteriocin polypeptide, and combining the bacteriocin polypeptides to prepare a bacteriocin complex.

23. The method of claim 1, wherein the Class I or Class II bacteriocin is a Class I bacteriocin, wherein optionally the method comprises harvesting the bacteriocin polypeptide and a further step (d) of cleaving the inactive precursor to remove the leader sequence; and/or wherein the Class I bacteriocin is a lantibiotic.

24-25. (canceled)

26. The method of claim 23, wherein the lantibiotic is selected from the group consisting of nisin, bisin, lacticin, subtilin, epicidin, epidermin, epilancin, salvaricin, sublancin, carnocin, variacin, cypemycin, gallidermin, mersacidin, actagardine, cinnamycin, duramycin, ancovenin, actagardine, cytolysin, staphylococcin and mutacin,

27. The method of claim 23, wherein the nucleic acid molecule comprises
(a) a synthetic operon comprising:
(i) a promoter;
(ii) a structural gene encoding the bacteriocin; and
(iii) nisB, nisC and nisT genes, wherein preferably the bacteriocin is nisin; or
(b) a synthetic operon comprising:
(i) a promoter;
(ii) a structural gene encoding the bacteriocin polypeptide which is a chimeric bacteriocin polypeptide which comprises the leader sequence of nisin;
(iii) nisB, nisC and nisT genes, wherein preferably the flavulin.

28. (canceled)

29. A product obtainable by a method as claimed in claim 1.

30-31. (canceled)

32. A strain of coryneform bacteria which has been modified to express a Class I or Class II bacteriocin polypeptide, wherein the bacteriocin polypeptide is an inactive precursor, and/or said strain is not susceptible to said bacteriocin and/or said bacteriocin polypeptide is a component polypeptide of a multi-peptide bacteriocin and said modified strain does not produce all other component polypeptides required to make a functional bacteriocin, wherein preferably said modified bacterial strain a) does not contain a gene which provides the bacterial strain with immunity to the bacteriocin or b) contains a constitutively expressed gene which provides the bacterial strain with immunity to the bacteriocin.

33. The strain of claim 32, wherein said strain comprises an expression vector comprising a nucleic acid molecule comprising a synthetic operon comprising:
(i) a promoter controlling the expression of the following genes;
(ii) a structural gene encoding the bacteriocin polypeptide;
(iii) optionally, one or more genes encoding processing and/or transport proteins for production of said bacteriocin polypeptide; and/or
(iv) optionally, one or more genes which provide the bacterial strain with immunity to the bacteriocin;
wherein the nucleotide sequences of said genes are codon-optimised for expression in coryneform bacteria and wherein preferably the bacteriocin polypeptide is an inactive precursor.

34. The method of claim 1, wherein the bacterial strain is a species selected from Corynebacterium glutamicum, Corynebacterium efficiens, Corynebacterium ammoniagenes, Corynebacterium melassecola, Brevibacterium flavum, Brevibacterium lactofermentum, Brevibacterium divaricatum, Corynebacterium acetoacidophilum, Corynebacterium lilium, Corynebacterium casei, Corynebacterium stationis and Brevibacterium divaricatum.

35. The strain of claim 32, wherein the bacterial strain is a species selected from Corynebacterium glutamicum, Corynebacterium efficiens, Corynebacterium ammoniagenes, Corynebacterium melassecola, Brevibacterium flavum, Brevibacterium lactofermentum, Brevibacterium divaricatum, Corynebacterium acetoacidophilum, Corynebacterium lilium, Corynebacterium casei, Corynebacterium stationis and Brevibacterium divaricatum.
